(19) United States
(12) Patent Application Publication
(10) Pub. No.: US 2023/0313203 A1
(43) Pub. Date: Oct. 5, 2023

(54) FLUOROARABINO NUCLEIC ACID (FANA) APTAMERS THAT BIND SARS-2 RECEPTOR BINDING DOMAIN AND BLOCK BINDING TO THE ACE2 CELLULAR RECEPTOR

(71) Applicant: University of Maryland, College Park, College Park, MD (US)

(72) Inventor: Jeffrey J. DeStefano, Crofton, MD (US)

(21) Appl. No.: 18/193,033

(22) Filed: Mar. 30, 2023

Related U.S. Application Data
(60) Provisional application No. 63/362,325, filed on Mar. 31, 2022.

(51) Int. Cl. C12N 15/115; C12N 15/10; A61P 31/14

(52) U.S. Cl. CPC C12N 15/115; C12N 15/1048; A61P 31/14; C12N 2310/16; C12N 2310/322; C12N 2320/30

(57) ABSTRACT

The present disclosure generally relates to aptamers and, more specifically, to fluoroarabino nucleic acid (FANA) aptamers, that bind to the SARS-CoV-2 receptor binding domain (spike (“S”) protein) and thereby block binding to the angiotensin-converting enzyme 2 (ACE2) host cellular receptor. Such FANA aptamers have many applications including their use as antiviral drugs for treatment and prevention of diseases, such as COVID-19, resulting from SARS-CoV-2 infection.

This application claims benefit and priority to U.S. Provisional Application No. 63/362,325 filed on Mar. 31, 2022, which is incorporated herein by reference in its entirety.

### GOVERNMENT SUPPORT

This invention was made with government support under R01AI150480A and R21AI163816A awarded by the National Institutes of Health (NIH). The government has certain rights in the invention.

### TECHNICAL FIELD

The present disclosure generally relates to aptamers and, more specifically, to 2′-fluoro-arabino nucleic acid (FANA) aptamers, that bind to the S1 subunit of the SARS-CoV-2 spike (“S”) protein and thereby block binding to the angiotensin-converting enzyme 2 (ACE2) host cellular receptor. Such FANA aptamers have many applications including their use as antiviral drugs for treatment and prevention of diseases, such as COVID-19, resulting from SARS-CoV-2 infection.

### BACKGROUND

The coronavirus SARS-CoV-2 has had a devastating impact on society that will likely continue into the foreseeable future. It is the third coronavirus (SARS-CoV-1 and MERS being the other two) to emerge as a human pathogen in the past 17 years, raising the possibility that others will arise in the future [1,2]. Infection with SARS-CoV-2 requires interaction between the viral surface protein, spike (S), and a host “receptor” protein, angiotensin-converting enzyme 2 (ACE2) [3], that is expressed on type II alveolar cells [4] and ciliated cells in the human airway epithelium (HAE) [5], making these cells potentially vulnerable to infection. Reagents, such as antibodies that block this interaction have been successfully used to mitigate COVID-2 infections [6-8]. Thus, the development of novel therapeutics targeting SARS-CoV-2, or any additional viruses in general, are urgently needed.

### SUMMARY

The present disclosure relates to XNA nucleotide aptamers, and in particular, 2′-fluoro-arabino nucleic acid (FANA) nucleotide aptamers, and their use in treatment and/or prevention of viral infections. In a specific embodiment, the virus is a coronavirus such as SARS-CoV-2. While the disclosure below is directed to SARS-CoV-2 FANA aptamers that bind specifically to the SARS-CoV-2 spike (S) protein, it is understood that said disclosure can be applied equally as well to other viruses including, but not limited to, those having corresponding spike (S), proteins.

In an embodiment, SARS-CoV-2 FANA nucleotide aptamers are provided that bind to the RBD (Arg319-Phe541) and the larger S1 domain (Val16-Arg685) of the 1272 amino acid viral S protein, thereby blocking interaction between the viral S protein and the ACE2 cell receptor.

In a non-limiting embodiment, the SARS-CoV-2 FANA aptamers are derived from, the FAN-R8-17 (SEQ ID NO: 3) and FANA-R8-9 (SEQ ID NO: 11) sequences. In an embodiment, the FANA aptamers comprise the 40 nucleotide central sequence of FANA-R8-5/1-40 (SEQ ID NO: 2), FANA-R8-17/1-40 (SEQ ID NO: 4), FANA-R8-28/1-40 (SEQ ID NO: 6), FANA-R8-16/1-40 (SEQ ID NO: 8), FANA-R8-23/1-40 (SEQ ID NO: 10), FANA-R8-9/1-40 (SEQ ID NO: 12), FANA-R8-10/1-40 (SEQ ID NO: 14), FANA-R8-12/1-40 (SEQ ID NO: 16), and FANA-R8-22/1-40 (SEQ ID NO: 18), or fragments thereof. Said SARS-CoV-2 FANA aptamers may further comprise the fixed DNA primer region 5′-AAAAGGTAGTGCTGAATTCG-3′ (SEQ ID NO: 19) at the 5′ end and the fixed FANA primer region 5′-UUCGCUAUCCAGUUGGCCU-3′ at the 3′ end (SEQ ID NO: 20).

In an embodiment, provided SARS-CoV-2 FANA aptamers include those depicted in FIG. 2 (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17) or fragments thereof.

In a non-limiting embodiment, the 5′ and/or 3′ fixed primer sequences may comprise DNA sequences or FANA nucleotide sequences. The 5′ and/or 3′ fixed primer sequences may comprise DNA sequences or FANA nucleotide sequences having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 19 and/or 20, or fragments thereof. Further, in another embodiment the 5′ or 3′ fixed primer sequences may be truncated versions of SEQ ID NO: 19 and/or SEQ ID NO: 20. Such truncations included those of SEQ ID NO: 22, SEQ ID NO: 23, SEQ ID NO: 24, SEQ ID NO: 25 and SEQ ID NO: 26 that retain the ability to bind equivalently as FANA-R8-9 (SEQ ID NO: 11) to the S1 RBD protein, for example.

Accordingly, in an embodiment, the provided SARS-CoV-2 FANA aptamers comprises: 5′AAAAGGTAGTGCTGAATTCG(N)₄₀UUCGCUAUCCAGUUGGCCU-3′ (SEQ ID NO: 21) wherein the (N)₄₀ represents the 40-nucleotide central sequence. In yet another embodiment, the SARS-CoV-2 FANA aptamers include those comprising SEQ ID NOs: 1, 3, 5, 7, 8, 9, 11, 13, 15, 17, 22, 23, 24, 25 or 26 or a FANA aptamer sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

In yet another embodiment, the SARS-CoV-2 FANA aptamers include those comprising a random region of SEQ ID NOs: 2, 4, 6, 8, 10, 12, 14, 16, or 18, or a FANA aptamer sequence comprising a random region having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

The SARS-CoV-2 FANA aptamers provided herein bind with high affinity to targets and are RNase resistant. The disclosed FANA aptamers may further include one or more modifications that, for example, increase the stability of the aptamer. The FANA aptamers may also be conjugated to a therapeutic compound thereby providing a delivery device for targeting of the therapeutic compound to the FANA aptamer target.

In still another aspect, a method is provided for preparation of the aptamers provided herein. The preparation methods according to the present disclosure may be performed through nucleotide synthesis technology known in the art. Such methods are performed using 2′-deoxy-2′-fluoro-arabino nucleotides (faATP, faCTP, faGTP, faUTP) which result in FANA aptamer synthesis. Such methods include, for example, enzymatic synthesis using available natural and modified DNA polymerases and chemical synthesis using available FANA phosphoramidites. In a specific embodiment, DNA polymerases, such as the thermostable polymerase D4K and the BST DNA polymerase I, can be used for synthesis. In another embodiment, chemical synthesis using FANA phosphoramidites may be used for scaling up synthesis of the disclosed FANA aptamers.

Another aspect of the present disclosure provides an anti-viral composition comprising a FANA aptamer, as disclosed herein, as an active ingredient. As used herein, the term “anti-viral composition” refers to a composition able to prevent infection or re-infection with a viral pathogen. In a non-limiting embodiment, the virus is SARS-CoV-2, and the antiviral composition is able to reduce the severity of symptoms or eliminate the symptoms of COVID-19, or substantially or completely removing COVID-19 or the disease caused by SARS-CoV-2 infection. Thus, the anti-viral compositions disclosed herein may be administered prophylactically to a subject, e.g., a human, before infection with SARS-CoV-2, or may be therapeutically administered to subjects after infection with SARS-CoV-2.

The anti-viral compositions provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation. In a specific embodiment, the anti-viral composition is formulated for intranasal administration.

The anti-viral composition may contain a pharmaceutically acceptable carrier in order to be formulated. The carrier typically includes a diluent, an excipient, a stabilizer, a preservative, and the like. In a specific embodiment of the invention, the anti-viral composition is formulated for intranasal administration. In another embodiment, the anti-viral composition is formulated for systemic administration, for example, intramuscular administration.

Another aspect of the invention pertains to compositions comprising nanoparticles and the disclosed FANA aptamers. The nanoparticles can be created from biological molecules or from non-biological molecules. In some cases, the FANA aptamers are crosslinked to a polymer or lipids on the nanoparticle surface. In embodiments, the FANA aptamers are adsorbed onto the nanoparticle surface. In some embodiments, the disclosed FANA aptamers are adsorbed onto the nanoparticle surface and then crosslinked to the nanoparticle surface. In some embodiments, disclosed FANA aptamers are encapsulated into the nanoparticle. Such nanoparticles, or nanoliposomes may be incorporated into anti-viral compositions as disclosed below.

A method of treating a subject is provided that includes administering a disclosed anti-viral composition comprising a FANA aptamer, as described herein, to a subject in need thereof. In a non-limiting embodiment, the virus is SARS-CoV-2, and the antiviral composition is able to reduce the severity of symptoms or eliminate the symptoms of COVID-19, or substantially or completely removing COVID-19 or the disease caused by SARS-CoV-2 infection. Thus, the anti-viral compositions disclosed herein may be administered prophylactically to a subject, e.g., a human, before infection with SARS-CoV-2, or may be therapeutically administered to subjects after infection with SARS-CoV-2.

The disclosed anti-viral composition may be administered in a number of ways. For example, the disclosed anti-viral composition can be administered intramuscularly, intranasally, orally, intravenously, subcutaneously, transdermally (e.g., by microneedle), intraperitoneally, ophthalmically, sublingually, or by inhalation. In a specific embodiment, the anti-viral is administered intranasally.

In still yet another aspect, a diagnostic composition that employs the use of a viral specific FANA aptamer composition and methods for detecting the presence of a virus in a subject sample. In an embodiment, the virus is a coronavirus, and the FANA aptamer detects the presence of viral S protein, in a subject sample, and can be used to distinguish coronavirus-infected and uninfected subjects from each other by bringing the same into contact with a sample and measuring the extent of reaction there between. In particular, this composition may be useful to distinguish whether or not a patient with symptoms identical or similar to those of coronavirus disease is infected with coronavirus during the period of risk of onset of coronavirus disease. In a specific embodiment the coronavirus is SARS-CoV-2.

The present disclosure also provides for a method of identifying a FANA aptamer useful for use as an anti-viral composition. Such a method includes the use of systemic evolution of ligands by exponential enrichment (SELEX) for selection of FANA aptamers that bind to a target protein or nucleic acid of interest. Such a method comprises the steps of (i) incubating a FANA random pool of aptamers to the target of interest; (ii) washing any unbound FANA aptamers from the incubation mixture; and (iii) releasing the bound FANA from the target; (iv) reverse transcription of the released FANA to DNA; (v) conversion of reverse transcribed DNA to a FANA aptamer; and (vi) repeating the selection steps of (i) to (v), wherein the incubation of step (i) is done with the previously selected FANA(s), until a target binding FANA aptamer is identified with high binding affinity to the target. In an embodiment the target of interest is a viral protein. More specifically, the viral protein is the SARS-CoV2 RBD or S protein.

The present disclosure provides a kit that includes the aptamer anti-viral compositions, as described herein. In one specific aspect the kit further includes instructions for the treatment and/or prophylaxis of COVID-19. The anti-viral compositions may, if desired, be presented in a pack or dispenser device which may contain one or more-unit dosage forms containing the aptamer antiviral composition. In a specific embodiment, the dispenser may be one to be used for intranasal administration of the anti-viral composition. In a specific embodiment, the dispenser may be one to be used for intramuscular administration of the anti-viral composition. The pack may for example include metal or plastic foil, such as a blister pack. The pack or dispenser device may be accompanied by instructions for administration to subjects, especially humans.

### BRIEF DESCRIPTION OF THE FIGURES

FIG. 1 Structure of SARS-CoV-2 spike protein. The spike (S) protein has two major domains, subunit 1 (S1) and subunit 2 (S2). The receptor-binding domain (RBD) that binds to ACE2 is amino acid Arg319-Phe541 of the S1 domain. A 13 amino acid signal peptide (SP) is present at the start of the amino terminus, while the transmembrane domain (TM) is located near the C-terminus (amino acids 1214-1234). The numbering was taken from [44], and a more detailed representation can be found in that reference.

FIG. 2A-B. Sequence alignment using the MAFFT program for the random region (nucleotides 1-40) of recovered aptamers in the FANA-R8-17 and FANA-R8-9 lineages: (FIG. 2A) FANA-R8-5 to FANA-R8-23 are from the lineage containing FANA-R8-17, and FANA-R8-9 to FANA-R8-22 are from the FANA-R8-9 lineage. The fixed primer regions 5′-AAAAGGTAGTGCTGAATTCG-3′ at the 5′ end and 5′-UUCGCUAUCCAGUUGGCCU-3′ at the 3′ end are not shown in the alignment; (FIG. 2B) RNA fold program predicted structures of FANA-R8-9 and 17. Folded aptamers include primer regions. Red nucleotides are from the fixed primer regions at the 5′ and 3′ ends, while black nucleotides were derived from the random region of the starting material.

FIG. 3 Competition binding assay with SARS-CoV-2 RBD and S1 proteins. Samples contained 10 nM of 5′-32P end-labeled FANA-R8-9 aptamer and 10 nM of either RBD or S1 proteins (see below). Cold competitor (FANA-R8-9, FANA-ST (starting material for SELEX), or ACE2 protein) was added at 0, 1, 2, 4, 8, and 16-fold excess over labeled FANA-R8-9. RBD and S1 were omitted from the ACE2:FANA-R8-9 and ACE2:CoV2-RBD-1C (51 nt DNA aptamer). * All values are relative to the value for no competitor with RBD:FANA-R8-9 or S1:FANA-R8-9 (for S1 samples only).

FIG. 4. Example of an off-rate analysis of FANA-R8-9 from SARS-CoV-2 RBD, S1, and trimer proteins. Data was fit to a curve for single parameter exponential decay to calculate off-rate (k_{off}) and half-life (t_{1/2}). The experiment was repeated 3 times to yield k_{off} and t_{1/2} values shown in the insert table. The FANA-ST starting material bound very weakly in this assay with no significant level of bound material (using RBD) being measurable at the 20 min time point. * Values are relative to the value for bound material at time 0.

FIG. 5. ELISA assay to test the ability of antibodies and aptamers to block ACE2 binding to the RBD domain. Neutralizing RBD antibody (GenScript 6D11F2) was compared with FANA-R8-9 and FANA-ST (starting material) in an ACE2 ELISA assay (GenScript). * Percent inhibition was calculated based on positive and negative controls supplied by the manufacturer. The negative control produced a value of “0” in the assay and is not shown. See Materials and Methods for more details. ** Values are given in both μg/mL and nM. Note that each antibody contains two binding sites. The experiment was repeated with similar results.

FIG. 6A-B. FIG. 6A. Aptamer stability assay. 100 nM of radiolabeled FANA-R8-9 (79 nts) and DNA aptamer CoV2-RBD-1C (51 nts (46)) were incubated in 200 uL of D-MEM complete+10% FBS, and 1% penicillin/streptomycin) at 37° C. Twenty ul aliquots were removed at 0, 1, 2, 4, 8, and 24 h time points and run on a 10% PAGE denaturing gel. Lane ‘D’, a 20 uL aliquot was digested with DNaseI for 30 min at 37° C. as a control; (FIG. 6B) quantification of products. Gels were visualized and quantified using a phosphorimager. The level of full-length undegraded material was measured at each time point. The graph shows averages from three independent experiments with error bars representing the standard deviations. * Values were relative to the amount of material at time 0.

FIG. 7. Testing of the ability of FANA aptamers to block SARS-2 infection. FANA aptamer R8-9 but not controls, blocks infection of Wild Type (Wuhan strain) or Delta variant strain of SARS-CoV-2 virus in a Human Airway Epithelial (HAE) cell model. Cells from “Donor D” were differentiated to form a HAE model in culture. The R8-9 aptamer or various controls (a specific sequence of the same size chosen at random, or a pool of mixed sequences not selected for binding to target protein (scramble)) were mixed with live virus and cells at a concentration of 100 nM and infections were carried out for 3 days. Infected cell foci were labeled using a fluorescent antibody to SARS-CoV-2. Foci appear as dots on the darker background.

FIG. 8. Luciferase assay to test inhibition of pseudovirus entry on Vero E6 ACE2 cells. Increasing amount of R8-9 aptamer were added as described on the previous panel. The FANA starting material control at 50 nM was approximately equivalent to the “0” aptamer sample (not shown). Error bars are average values from 3 sample wells+/−standard deviations.

FIG. 9. Determination of the Inhibitory Concentration 50% (IC₅₀) for the luciferase assay shown in FIG. 8. Numbers from FIG. 8 were plotted as log [Aptamer] vs. % inhibition to determine the IC₅₀. The experiment from FIG. 8 yielded a result of 11.8 nM while a value of 8.6+/−4.1 nM was determined from 4 independent experiments (average+/−standard deviation).

FIG. 10 A-D. LDH assays indicate aptamer R8-9 is not toxic in various donor-derived HAE cultures. (FIG. 10A; Donor A, FIG. 10B; Donor F, FIG. 10C; Donor E, FIG. 10D; Donor B). Note: higher absorbance corresponds to more released LDH which is indicative of more cell death. % cell death=(sample abs/avg cell lysis abs)*100. Negative control—no cells (sets background). Normal—Cells, no treatment. PBS—Cells treated with PBS buffer that aptamer was resuspended in. Cell lysis control-lysed cells, set maximum detection of cell death.

FIG. 11A-D. TEER resistance measurements indicate aptamer R8-9 is not toxic in various donor-derived HAE cultures. (FIG. 11A; TEER Donor A, FIG. 11B; TEER Donor F, FIG. 11C; TEER Donor E, FIG. 11D; TEER Donor B). Note: Low resistance results from decreased integrity of the cell monolayer which corresponds to more cell death. Negative control—no cells (sets background). Normal—Cells, no treatment. PBS—Cells treated with PBS buffer that aptamer was resuspended in. Cell lysis control-lysed cells, set lowest level of resistance indicative of dead cells which cannot block current flow.

### DETAILED DESCRIPTION

The present disclosure provides FANA aptamers, which bind with high affinity to a desired target. FANA aptamers possess many attractive features including, for example, their smaller size, flexible structure, low immunogenicity, ability to be modified and conjugated to improve their pharmacokinetics profile and prolong half-life in vivo. Furthermore, FANA aptamers have many manufacturing benefits as the manufacture of FANA aptamers is completely an in vitro process, relying on enzymatic or chemical based synthesis procedures. Moreover, the tendency of FANA aptamers to form complementary base pairs confers additional benefits, as the function of FANA aptamers can be modulated in vivo using “antidote” oligonucleotides, which disrupt aptamer function by base pairing with the active motifs of the FANA aptamers.

Such FANA aptamers have many applications including replacement of antibodies in biochemical assays (e.g. ELISA), utilization as biosensors (including a recent rapid test for SARS-CoV-2 (9)), and as tools for studying virus molecular biology, and development of antiviral drugs (10-18). FANA aptamers have shown potent antiviral activity and low toxicity in cell culture (14, 19-29) and they are among the most potent inhibitors of protein activity in vitro (30-32). In an embodiment, the FANA aptamer technology disclosed herein may be used as a preventive, or treatment, of any diseases resulting from infection with a variety of different pathogenic organisms including, for example, viruses, bacteria and fungi.

In a specific embodiment, the FANA aptamers disclosed herein may be used as a preventive, or treatment, of viral infection. The virus may be a DNA virus or RNA virus. The virus may be selected from the following family of viruses Adenoviridae, Anelloviridae, Arenaviridae Astroviridae, Bornaviridae, Bunyaviridae, Caliciviridae, Coronaviridae, Filoviridae, Hepadnaviridae, Hepevirdae, Herpesviridae, Orthomyxoviridae, Papillomaviridae, Paramyxoviridae, Parvoviridae, Picobirnaviridae, Picobirna, Picornaviridae, Pneumoviridae, Polyomaviridae, Poxviridae, Reoviridae, Retroviridae, Rhabdoviridae, Togaviridae, and Delta.

In another embodiment, the virus is a coronavirus. As used herein, the term “coronavirus” is meant to include all microorganisms classified and identified as coronavirus. There are hundreds of coronaviruses, most of which circulate among such animals as pigs, camels, bats and cats. Coronaviruses are a large family of viruses that usually cause mild to moderate upper-respiratory tract illnesses, such as the common cold. However, coronaviruses have emerged from animal reservoirs over the past two decades to cause serious and widespread illness and death. Such coronaviruses include, for example, SARS coronavirus (SARS-CoV) causing severe acute respiratory syndrome (SARS), MERS coronavirus (MERS-CoV) causing Middle East respiratory syndrome (MERS) and SARS-CoV-2 causing coronavirus disease 2019 (COVID-19). While the disclosure below is directed to SARS-CoV-2 FANA aptamers, it is understood that said disclosure can be applied equally as well to other viruses, coronaviruses, and their variants.

The causative agent of COVID-19, SARS-CoV-2, gains access to cells through interactions of the receptor binding domain (RBD) on the viral S protein with angiotensin converting enzyme 2 (ACE2) on the surface of human host cells. The present disclosure relates to novel FANA aptamers that interact with the SARS-CoV-2 viral receptor binding domain (RBD) or the S protein thereby inhibiting the binding of the virus to the angiotensin-converting enzyme 2 (ACE2) on the surface of human host cells.

In an embodiment, FANA nucleotide aptamers are provided that bind to the RBD (Arg319-Phe541) and the larger S1 domain (Val16-Arg685) of the 1272 amino acid viral S protein, thereby blocking interaction between the viral S protein and the ACE2 cell receptor.

In a non-limiting embodiment, the SARS-CoV-2 FANA aptamers are derived from, the FAN-R8-17 (SEQ ID NO: 3) and FANA-R8-9 (SEQ ID NO: 11) sequences. In another embodiment, FANA aptamers are provided comprising the 40 nucleotide central sequence of FANA-R8-5/1-40 (SEQ ID NO: 2), FANA-R8-17/1-40 (SEQ ID NO: 4), FANA-R8-28/1-40 (SEQ ID NO: 6), FANA-R8-16/1-40 (SEQ ID NO: 8), FANA-R8-23/1-40 (SEQ ID NO: 10), FANA-R8-9/1-40 (SEQ ID NO: 12), FANA-R8-10/1-40 (SEQ ID NO: 14), FANA-R8-12/1-40 (SEQ ID NO: 16), and FANA-R8-22/1-40 (SEQ ID NO: 18), or fragments thereof. Said SARS-CoV-2 FANA aptamers may further comprise the fixed DNA primer region 5′-AAAAGGTAGTGCTGAATTCG-3′ (SEQ ID NO: 19) at the 5′ end and the fixed FANA primer region 5′-UUCGCUAUCCAGUUGGCCU-3′ at the 3′ end (SEQ ID NO: 20).

In an embodiment, provided SARS-CoV-2 FANA aptamers are those depicted in FIG. 2 (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17).

In a non-limiting embodiment, the 5′ and/or 3′ fixed primer sequences may comprise DNA sequences or FANA nucleotide sequences. The 5′ and/or 3′ fixed primer sequences may comprise DNA sequences or FANA nucleotide sequences having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 19 and 20, or fragments thereof. Further, in another embodiment the 5′ or 3′ fixed primer sequences may be truncated versions of SEQ ID NO: 19 and/or SEQ ID NO: 20. Such truncations included those of SEQ ID NO: 22, SEQ ID NO: 23, SEQ ID NO: 24, SEQ ID NO: 25 and SEQ ID NO: 26 which retain the ability to bind equivalently as FANA-R8-9 (SEQ ID NO: 11) to the S1 RBD protein, for example.

Accordingly, in an embodiment, the provided SARS-CoV-2 FANA aptamers comprises: 5′AAAAGGTAGTGCTGAATTCG(N)₄₀UUCGCUAUCCAGUUGGCCU-3′ (SEQ ID NO: 21) wherein the (N)₄₀ represents the 40-nucleotide central sequence.

In yet another embodiment, the SARS-CoV-2 FANA aptamers include those comprising SEQ ID NOs: 1, 3, 5, 7, 8, 9, 11, 13, 15, 17, 22, 23, 24, 25 or 26, or a FANA aptamer sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

In yet another embodiment, the SARS-CoV-2 FANA aptamers include those comprising a random region of SEQ ID NOs: 2, 4, 6, 8, 10, 12, 14, 16, or 18, or a FANA aptamer sequence comprising a random region having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

The term “identity” or “sequence identity” is known in the art and refers to a relationship between two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference).

The nucleotide aptamer sequences disclosed herein can further be used as a “query sequence” to perform a search against public databases to, for example, to identify other, or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to proteins of the disclosure. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

The present disclosure provides aptamer oligonucleotides that are at least 15 nucleotides in length. In one embodiment, at least 20 nucleotides in length. In one embodiment, at least 25 nucleotides in length. In one embodiment, at least 30 nucleotides in length. In one embodiment, at least 35 nucleotides in length. In one embodiment, at least 40 nucleotides in length. In one embodiment, at least 45 nucleotides in length. In one embodiment, at least 50 nucleotides in length. In one embodiment, at least 60 nucleotides in length. In one embodiment, at least 80 nucleotides in length. In one embodiment, at least 90 nucleotides in length. In one embodiment, at least 100 nucleotides in length.

The present disclosure further provides aptamer oligonucleotides 30-100 nucleotides in length, 40-90 nucleotides in length, 50-80 nucleotides in length, 50-60 nucleotides in length, 60-70 nucleotides in length, 70-80 nucleotides in length, or 80-100 nucleotides in length.

Included are FANA aptamers, as disclosed above, but which contain nucleotide substitutions or deletions, and which are nevertheless able to interact with the SARS-CoV-2 viral RBD domain (Arg319-Phe541) and/or the larger S1 domain (Val16-Arg685) and thereby inhibit binding of the virus to the ACE2 on the surface of human host cells when included in an anti-viral composition.

In an embodiment, techniques effective in improving the stability and prolonging serum half-life of the FANA aptamer may be employed. In an embodiment, modified nucleotide bases, sugar rings, or phosphates may be included in the aptamer structure to result in, for example, “slow off-rate modified FANA aptamers” with enhanced binding affinities and kinetics compared to conventional aptamers.

The aptamers provided herein may include stabilized derivatives with alterations on the sugar rings, including 2′-fluoro (2′-F) ribose, 2′-amino (2′-NH2) ribose, 2′-O-methyl (2′-OMe) ribose, or locked nucleic acids (LNAs), bridging the 2′- and 4′-ribose positions covalently). Aptamers with the dT bases replaced with dU bases modified at the 5-position of the heterocyclic base may be used. Hydrophobic replacement at the 5-position may also be utilized to increase stability.

Phosphate linkage modifications can also be introduced into aptamers for stabilizing the chains of nucleic acids by replacing conventional phosphate (PO) backbones with sulfur-containing phosphate ester bonds, including phosphorothioate (PS) bonds and phosphorodithioate (PS2) bonds. Oligonucleotide chains composed of L-nucleotides, as opposed to the natural sequences composed of D-nucleotides may be employed forming left-handed helices instead of the conventional right-handed helices.

Techniques designed to protect aptamers from nuclease degradation include, for example, the modification of (i) sites at either end of the oligonucleotide, (2) the sugar ring, and (3) the phosphodiester backbone. Such modifications include capping the 3′ end with inverted thymidine. A number of modifications are possible on riboses, such as 2′-F, 2′-NH2, or 2′-OMe substitutions, isonucleotide substitutions, and replacement of ribonucleotide analogues with locked nucleic acid (LNA, linking 2′-O and 4′-C of the ribose with a methylene bond). Modifications also include, for example, LNA, 2′-OMe, 2′-fluor RNA, 1′, 5′-anhydrohexitol nucleic acids (HNA), and 2, altritol nucleic acids (ANA) modifications. Modification of the aptamer at the 5′ end with cholesterol, diacylglycerol (DAG) or a PEG chain (PEGylation) labeling may be used to increase the stability of the aptamers disclosed herein. Further, GalNAc or serum albumin modification of the aptamer may be used.

In another embodiment, the FANA aptamers disclosed herein may be conjugated to drug thereby providing a targeting drug delivery device for treatment of pathological conditions resulting from infections with viruses, bacteria or fungi. In such an instant, a FANA aptamer, identified for its ability to bind to a viral, bacterial or fungal target protein is conjugated to a drug useful for treatment or prevention of diseases resulting from infection with said pathogens. In a specific embodiment, the FANA aptamers are those disclosed herein that bind with high affinity to the SARS-CoV-2 viral RBD or S1 protein.

Methods of identification and production of FANA aptamers are provided. The preparation methods according to the present disclosure may be performed through nucleotide synthesis technology known in the art. Such methods include those performed using 2′-deoxy-2′-fluoro-arabino nucleotides (faATP, faCTP, faGTP, faUTP) which result in FANA aptamer synthesis. Such methods include, for example, enzymatic synthesis using available natural and modified DNA polymerases and chemical synthesis using available FANA phosphoramidites. In a specific embodiment, DNA polymerases, such as the thermostable polymerase D4K and the BST DNA polymerase I, can be used for synthesis.

In another embodiment, chemical synthesis using FANA phosphoramidites may be used for scaling up synthesis of the disclosed FANA aptamers. The FANA aptamers may be chemically synthesized based on the aptamer nucleotide sequence. Such chemical synthesis methods are well known in the art, and, for example, solid-phase synthesis technology, solution-phase synthesis technology and the like may be used, and commercially available automated DNA synthesizers and the like using these technologies may be used.

Methods of isolating and purifying the FANA aptamers are also well known in the art, and any known method may be used. Examples thereof may include ultrafiltration, gel filtration, ion exchange chromatography, affinity chromatography, HPLC, hydrophobic chromatography, isoelectric point chromatography, and combinations thereof. In a specific embodiment, the FANA aptamers, may be engineered to include a HIS-tag as a means for affinity chromatography.

The present disclosure provides nanoparticles comprising the FANA aptamers disclosed herein. Such nanoparticles can be natural or synthetic and may be incorporated into an anti-viral composition. They can be created from biological molecules or from non-biological molecules. In some cases, the FANA aptamer is crosslinked to a polymer or lipid on the nanoparticle surface. In embodiments, the FANA aptamer is adsorbed onto the nanoparticle surface. In some embodiments, the FANA aptamer is adsorbed onto the nanoparticle surface and then crosslinked to the nanoparticle surface. In some embodiments, the FANA aptamer is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phosphatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000), 1,2-distearoyl-sn-glycero-3-phosphoethanolamine-N-[methoxy(polyethylene glycol)-1000] (DSPE-PEG2000), and cholesterol.

In some embodiments, the FANA aptamer is coated on the nanoparticle using a crosslinking agent. In some embodiments, the FANA aptamer is adsorbed onto the nanoparticle surface. In some embodiments, the FANA aptamer is adsorbed onto the nanoparticle surface followed by covalent crosslinking of the FANA aptamer to the nanoparticle surface using a crosslinking agent. Crosslinking agents suitable for crosslinking the FANA aptamer to produce the nanoparticle, or to coat the FANA aptamer on the nanoparticle are known in the art.

The present disclosure provides an anti-pathogen composition comprising a FANA aptamer as an active ingredient. In an embodiment, the composition is an anti-viral composition. In a specific embodiment, the anti-viral composition comprises a SARS-CoV-2 binding FANA aptamer. As used herein, the term “anti-viral” refers to a composition able to prevent or reduce the infection or re-infection with a virus, such as a coronavirus, thereby reducing the severity of symptoms or eliminating symptoms of viral infection, or substantially or completely removing the disease caused by the virus by preventing viral interaction with the viral cell receptor. In an embodiment, the term “anti-viral” refers to a composition able to prevent or reduce the infection or re-infection with SARS-CoV-2, reducing the severity of symptoms or eliminating symptoms of COVID-19, or substantially or completely removing SARS-CoV-2 or the disease by SARS-CoV-2, in a human host. Thus, the anti-viral composition disclosed herein may be administered prophylactically to a subject, i.e., a human, before infection with SARS-CoV-2, or may be therapeutically administered to subjects after infection with SARS-CoV-2.

Useful delivery vectors for inclusion in the anti-viral compositions include biodegradable microcapsules, immuno-stimulating complexes (ISCOMs) or liposomes. Liposome vectors may also be used for delivery of nucleic acids or proteins. Such liposome vectors may be unilamellar or multilamellar vesicles, having a membrane portion formed of lipophilic material and an interior aqueous portion. The aqueous portion is used to contain the polynucleotide material to be delivered to the target cell. In general, the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the pertinent disclosures of which are incorporated by reference. Many other suitable liposome-forming cationic lipid compounds are described in the literature. See, e.g., L. Stamatatos, et al., Biochemistry 27:3917 3925 (1988); and H. Eibl, et al., Biophysical Chemistry 10:261 271 (1979). Alternatively, a microsphere such as a polylactide-co-glycolide biodegradable microsphere can be utilized. An aptamer nucleic acid is encapsulated or otherwise complexed with the liposome or microsphere for delivery of the nucleic acid to a tissue, as is known in the art.

The anti-viral compositions provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation.

The anti-viral composition may contain a pharmaceutically acceptable carrier in order to be formulated. The carrier typically includes a diluent, an excipient, a stabilizer, a preservative, and the like. Suitable examples of the diluent may include non-aqueous solvents such as propylene glycol, polyethylene glycol, vegetable oil such as olive oil and peanut oil, or aqueous solvents such as saline (for example, 0.8% saline), water (for example, 0.05 M phosphate buffer) containing a buffer medium, and the like, suitable examples of the excipient may include starch, glucose, lactose, sucrose, gelatin, malt, rice, flour, chalk, silica gel, sodium stearate, glycerol monostearate, talc, sodium chloride, anhydrous skimmed milk, glycerol, propylene, glycol, water, ethanol and the like, and suitable examples of the stabilizer may include carbohydrates such as sorbitol, mannitol, starch, sucrose, dextran, glutamate, and glucose, or proteins such as animal, vegetable or microbial proteins such as milk powder, serum albumin and casein. Suitable examples of the preservative may include thimerosal, merthiolate, gentamicin, neomycin, nystatin, amphotericin B, tetracycline, penicillin, streptomycin, polymyxin B and the like.

The provided anti-viral composition may be produced in an arbitrary unit dose. A unit dose refers to the amount of the active ingredient and the pharmaceutically acceptable carrier contained in each product packaged for use in one or more administrations to a subject, such as a human, and an appropriate amount of such active ingredient and carrier is an amount that may function as an anti-viral when inoculation with the anti-viral composition of the present disclosure is performed one or more times, and such an amount may be determined non-clinically or clinically as understood by those skilled in the art.

A method of treating a subject for coronavirus is provided that includes administering the disclosed coronavirus anti-viral composition to a subject in need thereof. A method of treating a subject for SARS-CoV-2 is provided that includes administering a disclosed anti-viral composition, comprising a SARS-CoV-2 FANA aptamer, to a subject in need thereof. Said subjects include any animal that serves as a host for a coronavirus. Said subject may be an animal under the care of a veterinarian. Said subject may be a mammal. Said subject may be a human.

The disclosed anti-viral compositions may be administered in a number of ways. For example, the disclosed anti-viral composition can be administered orally, intravenously, subcutaneously, transdermally (e.g., by microneedle), intraperitoneally, ophthalmically, vaginally, rectally, sublingually, or by inhalation. The anti-viral composition of the present disclosure may be administered in a controlled release system including, for example, a liposome, a transplantation osmotic pump, a transdermal patch, and the like.

Methods of systemic delivery include those methods known in the art that provide delivery of the active molecule (e.g., the FANA aptamer) to the circulatory system with distribution throughout the body. Systemic delivery methods include intramuscular, intravenous, subcutaneous, intraperitoneal, and oral. As will be understood, any method of systemic delivery is suitable for use as a means for vaccination. Particularly suitable methods of systemic delivery include intramuscular and intravenous delivery.

In a specific embodiment, the anti-viral compositions are formulated for intranasal administration. Intranasal administration of the anti-viral composition, if used, is generally characterized by inhalation. Compositions for nasal administration can be prepared so that, for example, the FANA aptamer can be administered directly to the mucosa (e.g., nasal and/or pulmonary mucosa).

Optionally, such intranasal anti-viral compositions may further advantageously comprise a mucoadhesive, such as cellulose derivatives, polyacrylates, a starch, chitosan, glycosaminoglycans, hyaluronic acid, and any combination thereof. The mucoadhesive may be present in the composition at about 0.1% to about 10% by weight. For example, the anti-viral composition can be formulated for intranasal delivery as a dry powder, as an aqueous solution, an aqueous suspension, a colloidal suspension, a water-in-oil emulsion, a micellar formulation, or as a liposomal formulation.

Methods for mucosal delivery include those methods known in the art that provide delivery of the composition to mucous membranes. Mucosal delivery methods include intranasal, intrabuccal, and oral. In some embodiments, the administration is intranasal. In these embodiments, the anti-viral composition may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. In some embodiments, the compositions of present invention can be delivered using a nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediately prior to use and squeezed or squirted into the nasal passages.

The dose of the anti-viral composition may be determined by a medical practitioner in consideration of patient characteristics such as age, weight, gender, symptoms, complications, and the incidence of other diseases. Further, the temporal interval of administration and the number of administrations may be determined in consideration of the dosage form that is used, the half-life of the active ingredient in the blood, and the like.

The exact amount of the anti-viral composition required may vary from subject to subject, depending on the species, age, weight and general condition of the subject and its mode of administration and the like. Thus, it is not possible to specify an exact amount for every composition. However, an appropriate amount can be determined by one skilled in the art using only routine experimentation given the teachings herein. For example, effective dosages and schedules for administering the anti-viral compositions may be determined empirically, and making such determinations is within the skill in the art. The dosage ranges for the administration of the anti-viral compositions are those large enough to produce the desired effect in which the symptoms of the disorder are affected. The dosage should not be so large as to cause adverse side effects, such as unwanted cross-reactions, anaphylactic reactions, and the like. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The terms “treat/treating/treatment” and “prevent/preventing/prevention” as used herein, refers to eliciting the desired biological response, i.e., a therapeutic and prophylactic effect, respectively. In accordance with the present disclosure, the therapeutic effect includes one or more of a decrease/reduction in the severity of the disease (e.g., a reduction or inhibition of infection), a decrease/reduction in symptoms and disease related effects, an amelioration of symptoms and disease-related effects, and an increased survival time of the affected host, following administration of the anti-viral composition. A prophylactic effect may include a complete or partial avoidance/inhibition or a delay of infection, and an increased survival time of the affected host, following administration of the anti-viral composition.

Toxicity or efficacy of FANA aptamer components can be determined by standard procedures in cell cultures or experimental animals. Data obtained from cell culture assays and laboratory animal studies can be used in formulating a range of dosages for use in humans. The dosage of such components lies, for example, within a range of administered concentrations that include efficacy with little or no toxicity. The dosage may vary within this range depending upon the dosage form employed and the route of administration utilized.

Still a further aspect pertains to a diagnostic method of detecting a coronavirus-specific antigen, for example, a coronavirus spike protein, in a subject derived sample. The method includes (a) contacting a sample with a FANA aptamer composition for detecting a coronavirus-specific antigen, and (b) detecting the FANA aptamer/antigen complex. In an embodiment, the sample in step (a) is a nasal swab.

The diagnostic method is able to distinguish coronavirus-infected and uninfected subjects from each other by bringing the FANA aptamer into contact with a sample and measuring the extent of reaction therebetween. Such a method may be useful to distinguish whether a patient with symptoms identical or similar to those of coronavirus disease is infected with coronavirus during the period of risk of onset of coronavirus disease. In a specific embodiment the coronavirus is COVID-19.

As used herein, the term “specific binding” means that the FANA aptamer specifically binds to a coronavirus-specific antigen, e.g., binds only to the antigen and does not substantially bind to other antigens. Here, the term “substantially” means that nonspecific binding, the extent of which is low, may occur, and such nonspecific binding may be removed by washing using a washing solution before detection of specific binding as described below.

As used herein, the term “sample” refers to a sample in which a coronavirus-specific antigen, especially a Spike protein antigen, may exist, and includes the blood, serum, plasma, saliva, tears, mucus, nasal mucus and the like.

In one aspect, the FANA aptamer is in the form of being dissolved in a soluble solution, for example, a carbonate buffer solution or a bicarbonate buffer solution, or in a lyophilized form. In another aspect, the FANA aptamer is fixed to a support, and examples of the solid support that may be used may include, but are not limited to, particles (resin beads, magnetic beads, metal microparticles, gold colloids, etc.), substrates (microtiter plates, glass substrates, silicon substrates, resin substrates, electrode substrates, membranes, etc.), and the like. Methods of fixing the FANA aptamer of the present disclosure to the support may include direct fixation through adsorption (e.g. coating) or indirect fixation using a linker that binds both to the protein and the support.

When the support is treated with a sample, the FANA aptamer containing support can form a complex with a coronavirus-specific antigen, especially a Spike protein specific antigen, contained in the sample. After induction of the complex formation, in order to remove non-specifically bound antigens or contaminants, washing may be performed using a washing buffer such as Tween 20 or a washing agent such as distilled water.

The FANA aptamer/antigen complex may be detected through any of various methods, whereby the presence or absence and/or the concentration of a FANA aptamer in the sample may be qualitatively and quantitatively determined. This will provide useful information as to whether the subject is infected with coronavirus. In a specific embodiment, the FANA aptamer may be labeled for detection of the aptamer/antigen binding.

A further aspect pertains to a diagnostic kit for detecting a coronavirus-specific antigen, especially a coronavirus spike protein. The detection kit of the present disclosure includes the FANA aptamer. The FANA aptamer contained in the kit may be provided in the form of being attached to or detached from a support or may be provided in a dissolved form in a soluble solution or in a lyophilized form.

The diagnostic kit may further include a detection agent for detecting a complex of the coronavirus-specific antigen, especially the coronavirus spike protein, in the sample and the FANA aptamer specifically binding to the coronavirus-specific antigen. The detection agent may be a secondary antibody conjugated with the label or enzyme described above.

Furthermore, the diagnostic kit may further include a carrier, a washing buffer, a diluted sample solution, an enzyme substrate, and a reaction stop solution, and may also include instructions to teach the method of use, including a method of analysis of the results, etc.

The present disclosure also provides for a method of identifying a FANA aptamer useful for use as an anti-viral composition. Such a method includes the use of systemic evolution of ligands by exponential enrichment (SELEX) for selection of FANA aptamers that bind to a target protein or nucleic acid of interest. Such a method comprises the steps of (i) incubating a FANA random pool of aptamers to the target of interest; (ii) washing any unbound FANA aptamers from the incubation mixture; and (iii) releasing the bound FANA from the target; (iv) reverse transcription of the released FANA to DNA; (v) conversion of reverse transcribed DNA to a FANA aptamer; and (vi) repeating the selection steps of (i) to (v), wherein the incubation of step (i) is done with the previously selected FANA(s), until a target binding FANA aptamer is identified with high binding affinity to the target. In an embodiment the target of interest is a viral protein. More specifically, the viral protein is the SARS-CoV2 RBD or S protein.

The anti-viral compositions may, if desired, be presented in a pack or dispenser device which may contain one or more-unit dosage forms containing the FANA aptamer. The pack may for example include metal or plastic foil, such as a blister pack. The pack or dispenser device may be accompanied by instructions for administration to subjects, especially humans. Associated with such container(s) can be a notice in the form prescribed by a governmental agency regulating the manufacture, use or sale of pharmaceuticals or biological products, which notice reflects approval by the agency of manufacture, use or sale for human administration. Thus, a kit is provided that includes the FANA aptamer, as described herein. In one specific aspect the kit further includes instructions for the treatment and/or prophylaxis of COVID-19.

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1

Infection with SARS-CoV-2 requires interaction between the viral surface protein, spike (S), and a host “receptor” protein, angiotensin-converting enzyme 2 (ACE2) [3], that is expressed on type II alveolar cells [4] and ciliated cells in the human airway epithelium (HAE) [5], making these cells potentially vulnerable to infection. Antibodies that block this interaction have been successfully used to mitigate COVID-2 infections [6-8].

In this report, the selection of FANA aptamers is described, which are short nucleic acid-based sequences that bind with high affinity to targets, to block the interaction between the S protein and the ACE2 receptor. FANA aptamers have many applications including the replacement of antibodies in biochemical assays (e.g., ELISA), utilization as biosensors (including a recent rapid test for SARS-CoV-2 [9]), and as tools for studying virus molecular biology, and development of antiviral drugs [10-18]. FANA aptamers have shown potent antiviral activity and low toxicity in cell culture [14,19-29], and they are among the most potent inhibitors of protein activity in vitro [30-32].

FANA aptamers are typically made from natural RNA or DNA using systematic evolution of ligands by exponential enrichment (SELEX) [33,34]. More recently, xeno-nucleic acids (XNA), which are nucleotide analogs with altered sugar, base, or phosphate backbones, have been employed in place of DNA or RNA. FANA aptamers offer strong promise as therapeutics and diagnostics, as they have low immunogenicity and, in the case of XNA aptamers, greater resistance to degradation [35-39]. The first production of 2′-fluoro-arabino nucleic acid (FANA) [40] XNA aptamers to proteins is described in [41,42]. These aptamers bind with exceptionally high affinity to targets and are completely RNase resistant. In this report, the generation of FANA aptamers to the receptor-binding domain (RBD) of the SARS-CoV-2 S protein that can block the interactions between the S protein and ACE2 receptor is described. Although this work is targeted for SARS-CoV-2, the established principles could potentially be used for other current or future viruses and the discovered FANA aptamers have potential not only as virus inhibitors, but also in diagnostics and as biosensors.

### Material and Methods

The 2′-deoxy-2′-fluoro-arabino nucleotides (faATP, faCTP, faGTP, faUTP) required for FANA synthesis were obtained from Metkinen Chemistry (Kuusisto, Finland). Deoxyri-bonucleotide triphosphates (dNTPs) were from Roche (Penzberg, Germany) or United States Biochemical (Cleveland, OH). Enzymes and buffers including Taq polymerase, T4 polynucleotide kinase (PNK), 10× ThermoPol buffer (Mg2+-free), and MgSO4 were from New England BioLabs (Ipswich, MA). Radiolabeled ATP (γ-³²P) was from PerkinElmer® (Waltham, MA). G-25 spin columns were from Harvard Apparatus (Holliston, MA). Miniprep DNA preparation kits were purchased from QIAGEN (Hilden, Germany). Ni-trocellulose filter disks (Protran BA 85, 0.45 μm pore size and 25 mm diameter) were from Whatman (Maidstone, United Kingdom). Magnetic beads (Dynabeads™ His-Tag Isolation and Pulldown) for selection were from Invitrogen (Waltham, MA). All DNA oligonucleotides were from Integrated DNA Technologies (IDT) (Coralville, IA). Thermostable polymerase D4K for FANA nucleic acid production was prepared as described and stored in aliquots at −80° C. [36]. The SARS-CoV-2 receptor binding domain (RBD)(Arg319-Phe541) (Wuhan strain) and human ACE2 protein (both C-terminal His-tagged) were from RayBiotech (Peachtree Corners, GA). The C-terminal His-tagged SARS-CoV-1 S1 protein (Met1-Arg667) was from SinoBiological (Beijing, China). The His-tagged (Val16-Arg685) and untagged (Gln14-Arg685) SARS-CoV-2 S1 proteins (both Wuhan strain), cPass™ SARS-CoV-2 Neutralization Antibody Detection kit, and monoclonal antibody (clone ID: 6D11F2) were from GenScript® (Piscataway, NJ). The C-terminal His-tagged SARS-CoV-2 S protein trimer construct (Wuhan strain) was from Antibodies-online Inc (Davis, CA). The C-terminal His-tagged RBD Delta variant (Lys452Arg, Thr478Lys, com-pared with Wuhan strain RBD) protein was from Abbexa (Cambridge, United Kingdom). All other chemicals were from Avantor (Radnor, PA), Fisher Scientific (Waltham, MA), or Sigma (St. Louis, MO).

End-Labeling of Oligonucleotides with T4 Polynucleotide Kinase

DNA oligonucleotides were 5′end-labeled in a 50 μL volume containing 10-250 pmol of the oligonucleotide of interest, 1× T4 PNK reaction buffer (provided by the manufacturer), 10 U of T4 PNK and 5-10 μL of (γ-³²P) ATP (3000 Ci/mmol, 10 μCi/μL). The labeling reaction was performed at 37° C. for 30 min according to the manufacturer's protocol. PNK enzyme was heat inactivated by incubating the reaction at 75° C. for 15 min. Excess radiolabeled nucleotides were then removed by centrifugation using a Sephadex G-25 column.

Selection of FANA Aptamers with SARS-CoV-2 RBD Using Magnetic Dynabeads™

The 79-nucleotide FANA random pool starting material (referred to as FANA-ST) for SELEX containing a 40-nucleotide central random region flanked at the 5′ end by 20 nucleotides of fixed sequence DNA (5′-AAAAGGTAGTGCTGAATTCG-3′), and at the 3′end by 19 nucleotides of fixed FANA sequence (5′-UUCGCUAUCCAGUUGGCCU-3′) (i.e., 5′-AAAAGGTAGTGCTGAATTCG(N)₄₀UUCGCUAUCCAGUUGGCCU-3′), was prepared as described previously [41]. About 200 pmol (˜1×10¹⁴ different sequences) of 5′ ³²P-labeled FANA starting pool was heated to 90° C. then snap-cooled on ice. The material was then incubated with 20 pmoles of SARS-CoV-2 RBD protein that had been attached to Dynabeads™ using the C-terminal His-tag. Incubations were in 200 μL PBS (137 mM NaCl, 2.7 mM KCl, 8 mM Na₂HPO₄, and 2 mM KH₂PO₄, pH 7.4) for 30 min with agitation at room temperature. The beads were washed 2× with 200 μL of PBS and the bound FANA material was removed by adding 200 μL of imidazole containing buffer (300 mM imidazole, 50 mM sodium phosphate pH 8.0, 300 mM NaCl, 0.01% Tween™-20) to the beads and heating for 5 min at 90° C., then removing the beads with a magnet. Bound FANA was recovered by precipitation with ethanol in the presence of 50 μg of glycogen. The material was reverse transcribed to DNA, amplified and converted to FANA for another round of selection as previously described [41]. The SELEX was stopped after round 8 as no further binding affinity increase was detected.

Sequence Analysis of FANA Products Recovered from Round 8

PCR products were prepared from FANA sequences recovered from round 8. The PCR material was cloned using a TOPO TA cloning kit from Life Technologies. DNA mini-preps were prepared, and the products were sequenced by Macrogen (Rockville, Maryland). The appropriate DNA oligonucleotide templates for some of the recovered sequences were synthesized, and generation of FANA material was performed as described [41].

### Determination of Apparent Equilibrium Dissociation Constant (KD,App) Using Nitrocellulose Filter Binding Assays

Standard reactions for K_{D,app} determinations were performed in 20 μL of PBS with 0.1 mg/mL BSA and 0.1 nM 5′³²P end-labeled aptamer. Increasing amounts of SARS-CoV-2 RBD or other proteins were diluted in the above buffer and were added in amounts that approximately flanked the KD,app value (estimated from initial experiments) for the aptamer. After 10 min at room temperature, the reactions were applied to a 25 mm nitrocellulose disk (0.45 μm pore, Protran BA 85, Whatman™) pre-soaked in filter wash buffer (25 mM Tris-HCl pH 7.5, 10 mM KCl). The filter was washed under vacuum with 5 mL of wash buffer at a flow rate of ˜1 mL/sec. Filters were then counted in a scintillation counter. A plot of bound aptamer vs. protein concentration was fit to the following equation for ligand binding, one-site saturation in SigmaPlot in order to determine the KD,app: y=Bmax(x)/(KD+x) where x is the concentration of protein, and y is the amount of bound aptamer.

### Competition Binding Assays

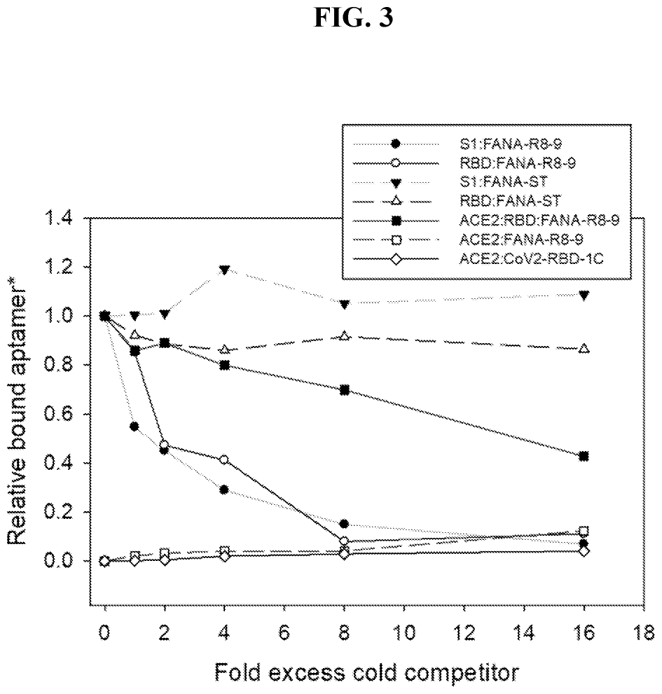

In total, 10 nM 5′³²P end-labeled aptamer was incubated at room temperature in PBS with various amounts of excess unlabeled competitor at 0-, 1-, 2-, 4-, 8-, or 16-fold excess over radiolabeled labeled aptamer. SARS-CoV-2 RBD or S1 protein was added to a final concentration of 10 nM. The total volume was 20 μL (in PBS). Incubations were continued for 1 h at room temperature. In competition reactions with the human ACE2 protein, the radiolabeled aptamer was mixed with ACE2 prior to the addition of SARS-CoV-2 RBD. Assays for measuring the binding of ACE2 to aptamers in the absence of RBD or S1 were also performed. The low level of binding to the aptamer in the absence of RBD was subtracted away from the result with radiolabeled aptamer, ACE2, and RBD to produce the final result (see FIG. 3). Samples were run over a nitrocellulose filter and washed and quantified as described above.

Dissociation Constant (koff) and Half-Life (t½) Determinations

For this, 5 nM (final concentration) 5′³²P end-labeled R8-9 aptamer was incubated for 10 min at room temperature in 90 μL PBS with 5 nM (final concentration) SARS-CoV-2 RBD, S1, or Trimer (as indicated). Unlabeled R8-9 aptamer was then added in a volume of 10 μL of PBS such that the final concentrations of unlabeled R8-9 were 125 nM (25-fold excess over labeled aptamer). For the SARS-CoV-2 Trimer, the concentration of unlabeled R8-9 was increased to 250 nM due to each trimer having 3 binding sites for the aptamer. A total of 10 μL aliquots were removed and filtered over nitrocellulose (see above) at “0”, 10, 20, 40, 60, 80, 100, and 120 min, or as indicated. Note that the time “0” was removed immediately upon the addition of unlabeled aptamer, but it is not a true time 0 as a few seconds passed before the material was filtered over nitrocellulose. Producing a time 0 sample by filtering the material prior to unlabeled aptamer addition produced results that were sometimes inconsistent with the remaining time points and produced experimental fits (see below) that were less accurate (based on r2 values). A background control was prepared by mixing 5 nM 5′³²P end-labeled R8-9 aptamer and 125 nM unlabeled aptamer in 9 ul of PBS, then adding 1 uL of SARS-CoV-2 RBD protein (final concentration 5 nM) and incubated for 10 min before processing. The dissociation constant was determined by fitting the data from a plot of aptamer bound to the filter vs. time, to an equation for a single 2-parameter exponential decay in SigmaPlot: y=ae−bx, where b is the dissociation constant (k_{off} in this case). The t½ value was determined from k_{off} using the following equation: t½=0.69/k_{off}.

### Binding Inhibitions Analysis

The ability of aptamers to block the association of SARS-CoV-2 RBD with ACE2 was measured with the cPass™ SARS-CoV-2 Neutralization Antibody Detection kit (GenScript®). For comparison, neutralizing monoclonal antibody (GenScript®, clone ID: 6D11F2) was also used. The manufacturer's suggested protocol for the kit was followed. Positive and negative controls were provided by the manufacturer. This protocol included an initial 30 min binding step with RBD and aptamer or antibody, followed by a 15 min incubation of the material with attached ACE2.

### Results

### Selection of FANA Aptamers Against the Spike Receptor Binding Domain (RBD)

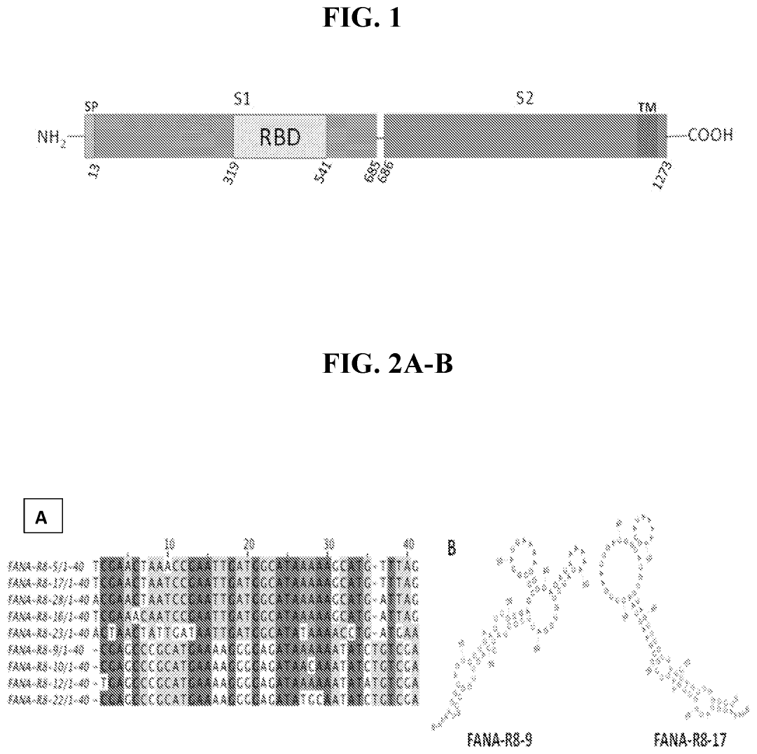

Aptamers were produced by a modified SELEX approach using mutated enzymes capable of converting between DNA and FANA [36]. The RBD domain of the (SARS-CoV-2) S protein was chosen as the target because aptamers that directly block S protein-ACE2 receptor interactions were desired, rather than those that bind S protein in other domains that may be less likely to block receptor binding. The 223 amino acid RBD (amino acid 319-541), comprises just a small portion of the S protein (1273 amino acids) (FIG. 1). It is part of the S1 subunit (amino acids 14-685) present on the outside of the viral envelope [43,44]. The C-terminal His-tagged RBD domain was attached to magnetic beads for the selection process (see, Materials and Methods). A total of 28 sequences were recovered from a limited number of clones after 8 rounds of selection. The recovered sequences were organized by sequence similarity into clusters using multiple alignment using fast Fourier transform (MAFFT) [45]. Sequences (7 total) from different clusters with diverse structures based on RNAfold analysis [46] were chosen for further testing. The FANA aptamer sequences are named based on the SELEX round (i.e., R8), and the number of the particular sequence clone. The two clusters containing the strongest RBD binding sequences (see Table 1, below) are represented by FANA-R8-9 and FANA-R8-17. These sequences are aligned with other recovered sequences from the same clusters (FIG. 2A) and the predicted structures of FANA-R8-9 and FANA-R8-17 are shown in FIG. 2B. The other sequences from these clusters had similar predicted structures. Aptamers from other clusters in Table 1 (FANA-R8-3, FANA-R8-7, and FANA-R8-15) that bound less tightly also had different predicted structures. Filter binding assays were used for measuring the apparent equilibrium dissociation constants (K_{D},app) to the RBD protein and the larger S1 portion of the SARS-CoV-2 spike protein (Val16-Arg685). All 7 tested FANA sequences bound to the RBD and S1 protein with ˜25-fold of greater binding affinity than the starting material (FANA-ST, Table 1). Aptamers FANA-R8-9, the closely related FANA-R8-22, and FANA-R8-17 bound the strongest, and FANA-R8-9 was chosen for further testing. This aptamer bound both the S1 protein and S protein trimer (a soluble form of the S trimer that is present on viral membranes) modestly more tightly than the RBD. A version of S1 without the His-tag bound with approximately the same affinity to FANA-R8-9 as tagged protein indicating that the His-tag played no role in aptamer binding. FANA-R8-9 was also tested for binding the Delta variant RBD protein. This protein differs from the Wuhan strain within the RBD domain at two positions, Lys452Arg and Thr478Lys. Interestingly, FANA-R8 bound about 10-fold better to this protein than the Wuhan strain RBD it was selected to. A similar phenomenon was observed for a DNA aptamer selected by the Wuhan RBD, which bound better to the UK variant [47]. The binding of FANA-R8-9 to the S1 protein from SARS-CoV-1 was also tested (Table 1). The spike proteins from these two viruses, which both use ACE2 as a receptor, are ˜76% identical at the amino acid level and ˜74% identical in the RBD domain [48]. The several-fold lower binding to S1 from SARS-CoV-1 demonstrates the high specificity of FANA-R8-9. The binding of RBD and S1 to DNA aptamer selected for RBD binding (CoV2-RBD-1C) was also tested. Aptamer CoV2-RBD-1C has a reported K_{D} for RBD of 5 nM, which suggests modestly tighter binding to RBD than FANA-R8-9 [49]. This aptamer bound weakly to the RBD construct but did bind to the S1 protein, albeit with lower affinity than the FANA aptamers. Differences between the published results and the results described herein may reflect the different affinity measurement techniques or different protein constructs.

Aptamer FANA-R8-9 was used in competition binding and off-rate analysis experiments. As expected, non-labeled FANA-R8-9 was able to compete with radio-labeled aptamer for binding to RBD or S1 (FIG. 3). However, non-labeled FANA-ST (starting material for SELEX selections) was unable to displace any FANA-R8-9 aptamer, even when added at 16-fold greater amounts. This confirms that FANA-R8-9 binds to RBD much more tightly than the starting material. In contrast, ACE2 protein was able to compete with FANA-R8-9 for binding to RBD. However, 2-fold excess cold FANA-R8-9 was as effective as 16-fold excess ACE2 in the competition. This indicates that FANA-R8-9 binds better to RBD than ACE2. ACE2 was also tested for binding to FANA-R8-9 and the CoV2-RBD-1C DNA aptamer [49]. Weak binding to ACE2 was detected for both aptamers, with twofold greater binding to FANA-R8-9 (FIG. 3).

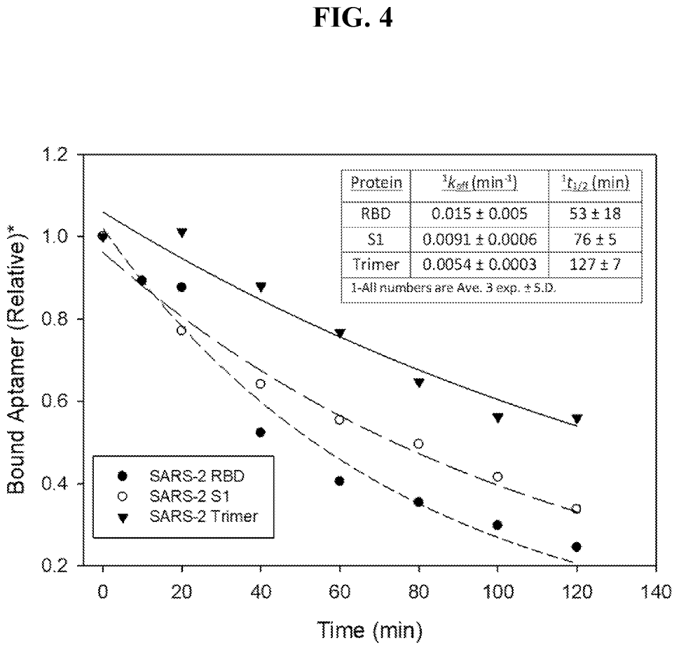

Off-rate analysis showed that FANA-R8-9 dissociated from RBD with a half-life of 53±18 min (Ave. 3 exp.±S.D., FIG. 4), demonstrating stable binding. Binding appeared to be more stable with SARS-CoV-2 S1 protein (76 t 5) and the S protein trimer (127±7 min). While the increase was not statistically significant for the S1 protein, it was for the S protein. More stable binding may be due to additional stabilizing contacts being available on the larger S protein. Another possibility for the S protein trimer is that there is a proximity effect since 3 RBD binding sites for the aptamer are presumably close together and an aptamer that dissociated from a site may quickly bind to a neighboring site.

### Functional Assessment of RBD-Binding Aptamers

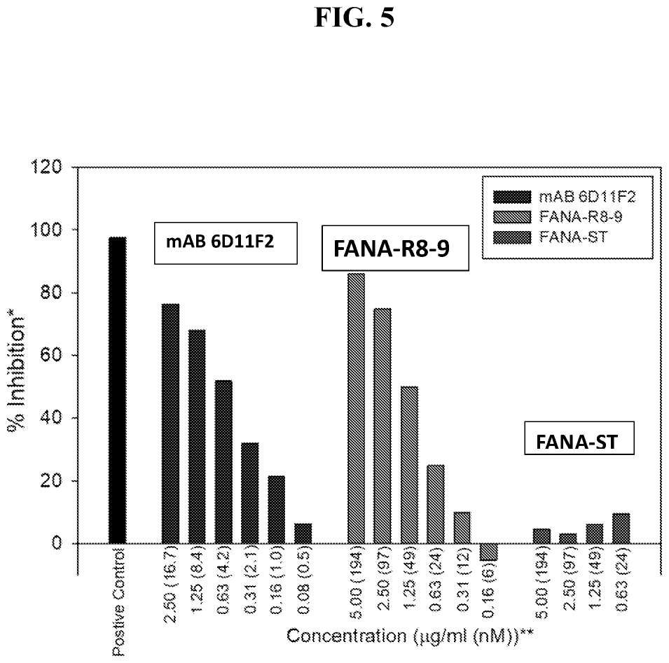

To measure the ability of aptamers to block the binding of the RBD to the ACE2 receptor, an ACE2 ELISA was used (FIG. 5). Aptamers were compared with an anti-SARS-CoV-2 RBD-neutralizing antibody (GenScript® clone ID. 6D11F2) and FANA-ST. On a weight basis (FIG. 5, see μg/mL amounts on X-axis), antibody 6D11F2 and FANA-R8-9 (as well as FANA-R8-22 (data not shown)) showed similar ability to block ACE2 binding (IC₅₀ ˜0.6 μg/mL for the antibody and 1.30±0.18 μg/mL (ave. 3 exp.±S.D.) for FANA-R8-9), while aptamer FANA-R8-17 was ˜threefold weaker, and FANA-ST showed no significant blocking of ACE2 binding. On a per molecule basis, the antibody was more effective as an inhibitor (FIG. 5, see “nM” amounts on X-axis). Considering that antibodies have two target binding sites vs. one on the aptamer, the antibody was about threefold better using this criterion.

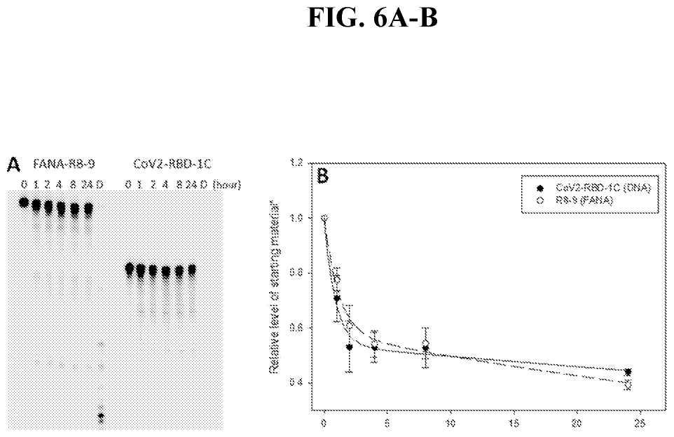

FANA-R8-9 was also tested for stability in serum-containing cell culture media (FIG. 6A). Both FANA-R8-9 and CoV2-RBD-1C DNA aptamer [49] remained intact for several hours and demonstrated similar decay rates (FIG. 6B). The slower rate of decay between 4 and 24 h may result from decreased activity of the degrading enzymes in the media.

This above disclosed example demonstrates the production of aptamers that can bind to and block the binding of the SARS-CoV-2 RBD to ACE2. The aptamers are unique, as they are made from FANA XNA, as opposed to previous DNA aptamer to the SARS-CoV-2 S protein. Binding, based on K_{D,app} analysis was comparable to previously reported DNA aptamers[47, 49, 50, 51, 52]. The aptamers were stable for several hours in cell culture media but did break down at a rate comparable to the tested DNA aptamer (FIG. 6).

Interestingly, a previously reported DNA aptamer (CoV2-RBD-1C) that bound with a K_{D} of 5.8±0.8 nM to RBD [49] did not bind strongly to the RBD in our system, although it did show binding to the S1 domain protein, albeit at a lower level than the FANA aptamers (Table 1). The RBD used for binding tests in our experiments was the same as the protein used for selection and included a C-terminal His-tag. Binding was also measured in solution using nitrocellulose filters. The CoV2-RBD-1C aptamer was measured using RBD attached to nickel beads. It is possible that the free His-tag (as opposed to the tag sequestered on beads) in our measurements interfered with binding. The S1 protein used in our measurements also contained a His-tag, but it is further away from the RBD domain due to the larger size of the protein. Other DNA aptamers to RBD have also been reported. Most report binding in the same low nM range, as the FANA aptamers described here [47,49,51]. This is in the same range as the reported interaction between ACE2 and the SARS-CoV-2 S protein (14.7 nM) and considerably tighter than SARS-CoV-1 S protein binding to ACE2 (325.8 nM) [53]. Therefore, it would be expected that these aptamers should be good competitors for ACE2 binding. In agreement with this observation, FANA-R8-9 was about as effective on a per weight basis as the neutralizing RBD-specific antibody used in this analysis (FIG. 5). As there are numerous variations in the type of aptamers that can be generated with different XNAs [54,55], perh

The FANA-R8-9 and other aptamers (Table 1) bound with low nM affinity to RBD, while previous FANA aptamers isolated in this lab to HIV reverse transcriptase (RT) and integrase (IN) bound with low pM affinity, ˜1000-fold tighter [41,42]. One reason for this is RT and IN are both natural nucleic acid binding proteins and already bind tightly to specific nucleic acids. It is more of a challenge to recover strong binding aptamers to proteins that do not naturally bind nucleic acids. However, this is not always the case. Aptamers to thrombin, for example, can bind with pM affinity and modified aptamer to VEGF, which is the target for aptamer therapy for macular degeneration, also show pM binding [56,57,58]. Several aptamers made using slow off-rate modified aptamers (SOMAs) technology that includes the addition of hydrophobic groups to nucleic acids bind tightly to targets, even those that are not natural nucleic acid-binding proteins [59]. Still, making aptamers is a “hit-or-miss” proposition, and there are no guarantees that aptamers that can bind more tightly than those reported here or by others can be found. It was notable that despite the modest low nM K_{D}'s of the FANA aptamers (Table 1), the observed off-rates were indicative of highly stable binding, especially for the more natural trimeric S protein (FIG. 4). Stable binding is likely a better predictor of potential therapeutic effect than a lower binding affinity [60].

Finally, the FANA aptamer has not yet been tested in virus neutralization assays. Aptamers that block interactions with the receptor, such as neutralizing antibodies, have the advantage of not having to enter cells to be effective. While DNA aptamers that bind the SARS-CoV-2 RBD have been shown to block virus infection [51,52], a recent report indicates that a DNA aptamer that binds to S1 but not in the RBD region can neutralize virus [50]. Interestingly, this aptamer did not appear to block virus binding to the ACE2 receptor. This suggests that even those aptamers that do not directly block binding may be able to inhibit replication.

### Example 2

Testing the Ability of FANA Aptamers to Block SARS-2 Infection.

Primary human airway epithelial (HAE) cells isolated from individual donor sources were differentiated into a pseudostratified epithelium and maintained at air-liquid interface. Cells from several de-identified donors were included in the experiments. HAE cultures were infected with approximately 104 plaque forming units of SARS-CoV-2 Wuhan or Delta strain. Infections were in the presence or absence of aptamer or controls at a concentration of 100 nM. The aptamer used was the R8-9 aptamer selected for high affinity binding to the Wuhan strain receptor binding domain (RBD). The two controls were a unique sequence randomly selected FANA of identical size from the starting material, and a control containing many different “random” sequences or identical size that were representative of the starting pool used to select the R8-9 aptamer. The infection was continued for 3 days with the aptamer and controls replaced each day to keep the concentrations “constant” as it was known that the aptamers break down over several hours. After 3 days the cells were processed as described on the next panel. Infection was detected using an antibody that can bind to the SARS-CoV-2 Nucleocapsid (N) protein. Antibody binding was confirmed by standard immunofluorescence. Staining results in a green dot in the panels where an infected cell is present.

Preparing infected cultures for staining. Cultures washed, permeabilized, and blocked in 3% BSA/PBS++. Subsequently stained with anti-N (overnight) and Alexa Fluor 488 secondary (2 hours). Images represent standardized exposure and display settings. “Median” or representative photo chosen of 3.

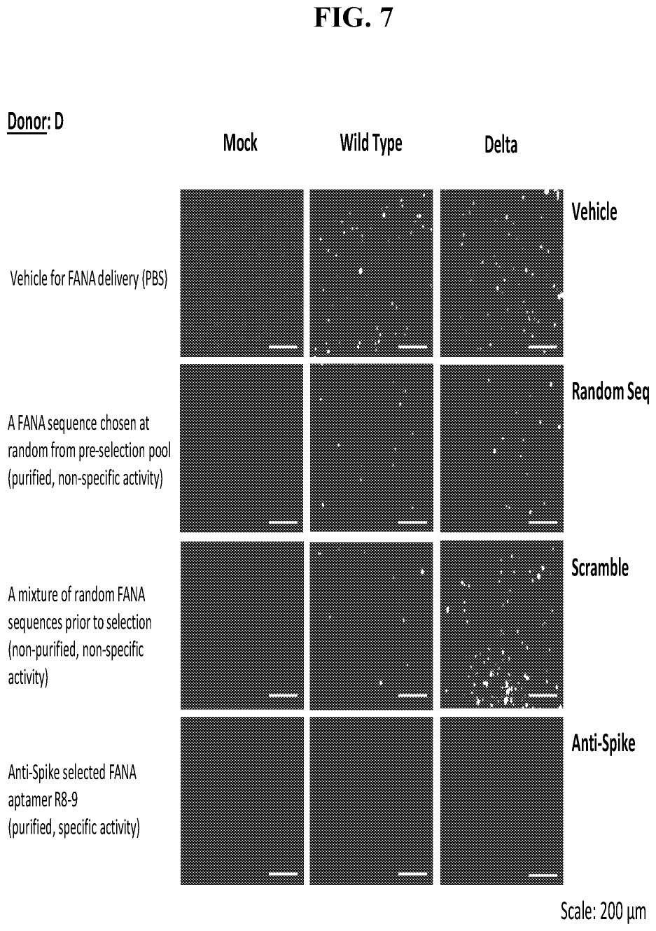

Results (see, FIG. 7, panel with immunostaining) indicated high levels of infection (Donor D is shown and is representative of the general trends with other donors although there were differences in the level of infection with different donors) of cells by both Wuhan and Delta virus. Wuhan virus showed a modest decline in infection with the controls and a dramatic decline to essentially undetectable levels of infection with aptamer R8-9. Delta was not strongly affected by the controls but was also essentially completely inhibited by R8-9. The experiments have been repeated using similar conditions and achieved consistent results.

### Example 3

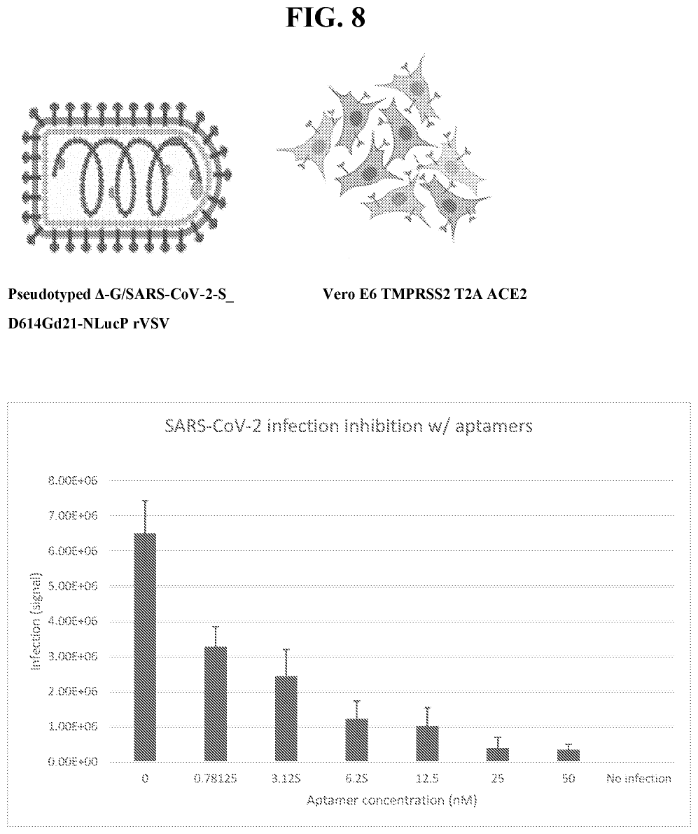

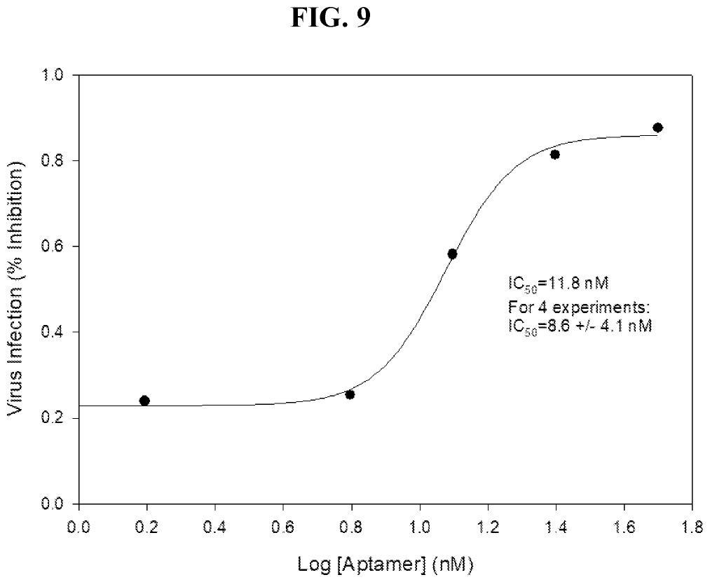

Fluoroarabino nucleic acid (FANA) aptamers inhibit viral entry in a SARS-CoV-2 pseudovirus assay. Pseudotyping has many uses in virology. “Pseudoviruses” are typically virus particles that carry the genome of a particular virus (either wild type or modified) while the outer membrane contains proteins from a different virus. The outer membrane proteins may allow entry of a viral genome into cell types that normally restrict entry, or they may enhance infection levels making some studies easier. They can also be used in experiments to study virus infection where the virus under study may be highly pathogenic and restricted for use in a particular lab setting. Another “twist” on this theme is to use the modified genome of a common virus as a viral entry “reporter” while decorating the outside of the viral particle with the receptor-binding protein from a different virus. Taking advantage of the available pseudovirus—Δ-G/SARS-CoV-2-S_D614Gd21-NLucP rVSV—the ability of FANA aptamer R8-9 to inhibit viral entry was examined. The pseudovirus contains a modified vesicular stomatitis virus (VSV) genome that codes for a nanoluciferase (nanoluc) reporter protein and has the Wuhan strain SARS-CoV-2 S protein in place of the VSV-G envelope protein. The pseudovirus expresses SARS-CoV-2 S protein on its surface, allowing entry into cells with ACE2 (the cellular receptor for SARS) on their surface. Expression of nanoluc in the infected cells allows quantification of the level of infection in the culture. Vero E6 TMPRSS2 T2A ACE2 cells were used as the virus target. Vero E6 cells are commonly used to monitor SARS-CoV-2 infection and these modified cells express high levels of ACE2 on their surface which enhances infection (Chang, C.-W.; Parsi, K. M.: Somasundaran, M.; Vanderleeden, E.; Liu, P.; Cruz, J.; Cousineau, A.; Finberg, R. W.; Kurt-Jones, E. A. A Newly Engineered A549 Cell Line Expressing ACE2 and TMPRSS2 Is Highly Permissive to SARS-CoV-2, Including the Delta and Omicron Variants. Viruses 2022, 14, 1369.https://doi.org/10.3390/v14071369). Experiments were conducted by infecting ˜40,000 cells per well with virus on 96-well plates at a Multiplicity of Infection (MOI) of 0.05, which empirically was found to yield a strong, but not saturating level of nanoluc activity. Infections were carried out by mixing virus with R8-9 aptamer, or control FANA material which was the starting material for aptamer selection and consisted of “random” sequence” material. This premixed material was used to infect cells which were quantified for nanoluc activity 24-hour post infection. Control FANA material was non-inhibitory to infections while R8-9 showed a concentration-dependent inhibition of viral infection. (See, FIG. 8 and FIG. 9)

### Example 4

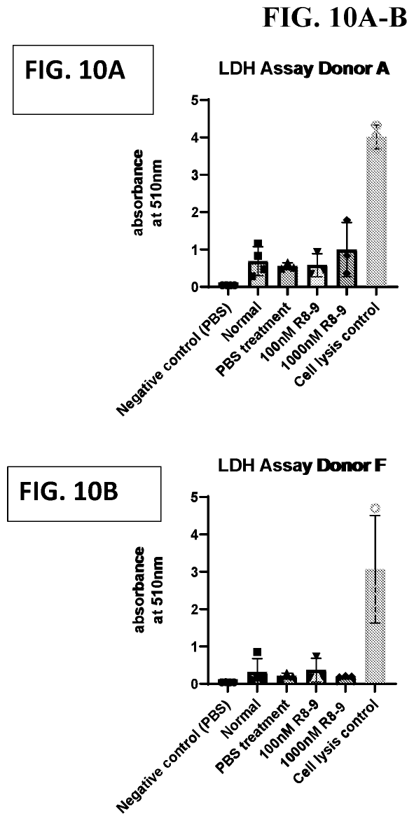

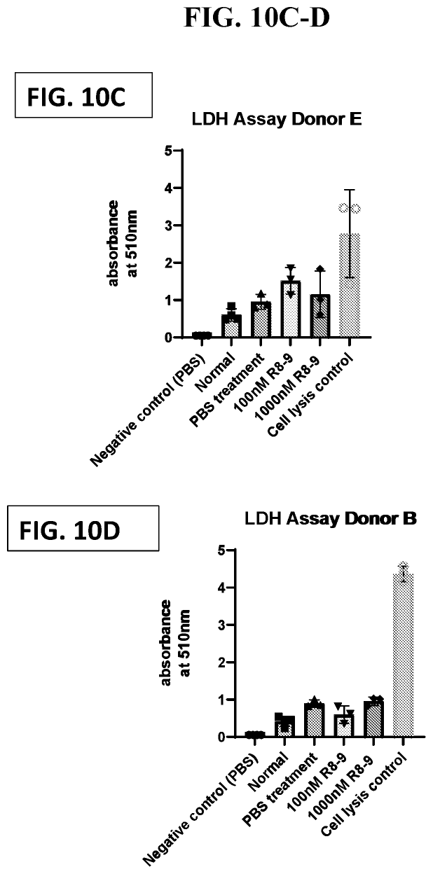

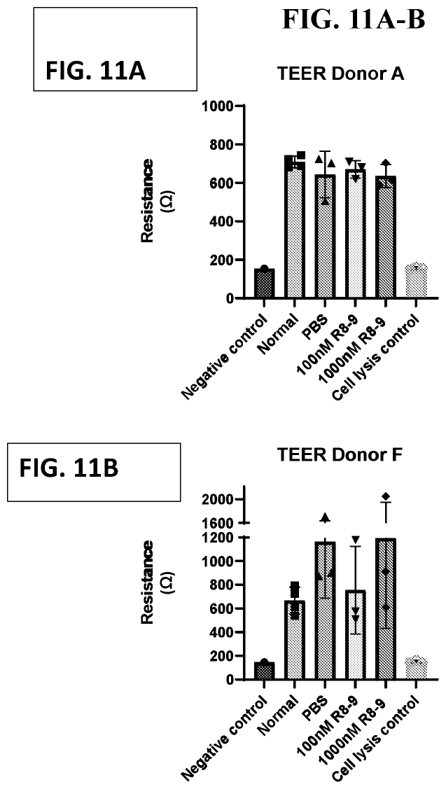

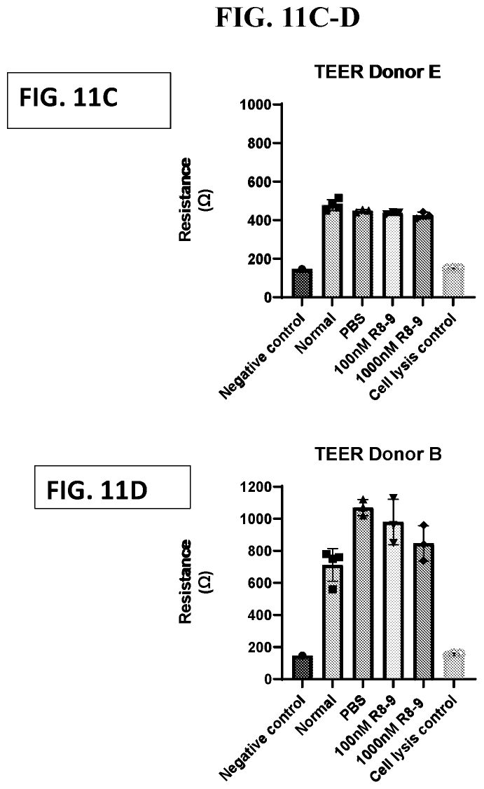

Fluoroarabino nucleic acid (FANA) aptamers to the SARS-CoV-2 receptor binding domain (RBD) are not toxic to cells. Therapeutics not only have to be effective in inhibiting the pathogen but must also be non-toxic to the host organism. As an initial step to testing organismal toxicity, the toxic effects of potential drugs in cell models are commonly performed. In the following experiments the prototype R8-9 aptamer was tested in the human airway epithelial (HAE) culture assay for potential cellular toxicity. Previous experiments demonstrated that in the HAE model, R8-9 was highly effective at inhibiting viral infection with Wuhan and Delta strains of SARS-CoV-2. At a concentration of 100 nM aptamer strong inhibition of Wuhan was observed and nearly complete inhibition of Delta. Toxicity assays were performed with 100 and 1000 nM aptamer with HAE cultures derived from several different de-identified donors using two approaches. Transepithelial-Transendothelial Electrical Resistance (TEER) is a validated, label-free and fast technique to measure the electrical resistance of a barrier tissue model (Srinivasan B, Kolli A R, Esch M B, Abaci H E, Shuler M L, Hickman J J. TEER measurement techniques for in vitro barrier model systems. J Lab Autom. 2015 April; 20(2):107-26). The electrical resistance is defined as the opposition to current flow in a circuit, it is therefore a quantitative parameter to evaluate integrity of a monolayer through its ionic conductance. A Millicell® ERS-2 Voltohmmeter was used for the tests (emdmillipore.com/US/en/life-science-research/cell-culture-systems/cell-growth/millicell-ers-2-voltohmeter/1Iub.qB.tjsAAAFBoHxb3.rd,nav). TEER values are in direct proportion to barrier integrity. For example, TEER rises as cells proliferate and it reaches the highest value at confluency, suggesting intactness of the cell layer. A reduced TEER value is an indicator of a compromised barrier that is losing integrity, due to compromised, dying, or dead cells for example. Therefore, TEER is a good method to evaluate drug toxicity in a monolayer culture system like HAE. The second approach utilized a lactate dehydrogenase (LDH) assay which is a standard procedure for quantifying cell death (Kumar P, Nagarajan A, Uchil PD. Analysis of Cell Viability by the Lactate Dehydrogenase Assay. Cold Spring Harb Protoc. 2018 Jun. 1; 2018(6)). These assays quantitatively measure LDH, a stable cytosolic enzyme that is released upon cell lysis. A standard kit from Promega (CytoTox 96® Non-Radioactive Cytotoxicity Assay) was used for these assays. Results from both assays indicated that FANA aptamer R8-9 showed no statistically measurable toxicity in any of the HAE donor cultures. (See, FIG. 10A-D and FIG. 11A-D)

TABLE 1
Apparent equilibrium dissociation constants (K_{D,app}) for
tested FANA aptamers

| | | | S1 (no his | ^{d}S protein |
|---|---|---|---|---|
| | ^{b}RBD K_{D,app} | ^{c}S1 K_{D,app} | tag) K_{D,app} | timer K_{D,app} |
| ªAptamer | (nM) | (nM) | (nM) | (nM) |
| ^{e}FANA-ST | ^{f}1695 ± 754 | | | |
| FANA-R8-3 | 68.1 ± 16.9 | 46.4 ± 2.8 | | |
| FANA-R8-5 | 26.4 | 16.6 | | |
| FANA-R8-7 | 62.8 ± 8.4 | 31.2 ± 11.7 | | |
| FANA-R8-9 | 23.5 ± 4.6 | 14.7 ± 3.9 | 29.6 ± 3.9 | 14.4 ± 4.6 |
| FANA-R8-9 | 1.4 ± 0.4 | | | |
| | (Delta Variant)^{g} | | | |
| ^{h}FANA-R8-9 | 19.6 ± 6.2 | | | |
| (D-MEM +10% FBS) | | | | |
| FANA-R8-15 | 43.3 ± 0.2 | 27.7 ± 5.3 | | |
| FANA-R8-17 | 29.5 ± 6.5 | 19.1 ± 7.5 | | |
| FANA-R8-22 | 21.3 | 13.3 | | |
| ⁱS1-SARS-CoV-1 | | 259 ± 161 | | |
| ^{j}CoV2-RBD-1C | 872 ± 359 | 105 ± 34 | | |

^{a}Aptamer sequences. Only the ~40 nt random regions of the ~79 nt aptamers are shown. In the full aptamer, this would be flanked by 5′-AAAAGGTAGTGCTGAATTCG-3′ at the 5′ end and, and 5′-UUCGCUAUCCAGUUGGCCU-3′ at the 3′ end, (see Methods)):
FANA-ST: 5′-NNNNNNNNNNNNNNNNNNNNNNNNNNNNNNNNNNNNNNNN-3′
FANA-R8-3: 5′-GTCGCGATTAACATTAAACCGCATAAAAAGGGTGGCCGGA-3′
FANA-R8-5: 5′-TCGAACTAAACCGAATTGATGGCATAAAAAGCATGTTTAG-3′
FANA-R8-7: 5′-ATTCTCGATTGATGGCATAAAAAGCATAGAATCGCAAGCA-3′
FANA-R8-9: 5′-CGAGCCCGCATGAAAAGGGGAGATAAAAAATATCTGTCGA-3′
FANA-R8-15: 5′-GGAGCTCGAACAGATGGGATAAAAAGCATAGCTCACCAAT-3′
FANA-R8-17: 5′-TCGAACTAATCCGAATTGATGGCATAAAAAGCATGTTTAG-3′
FANA-R8-22: 5′-CGAGCCCGCATGAAAAGGGGAGATATGCAATATCTGTCGA-3′
^{b}“RBD”: Receptor binding domain of SARS-CoV-2 spike protein (Wuhan strain unless otherwise stated) with C-terminal His-tag (see FIG. 1).
^{c}“S1”: S1 portion of SARS-CoV-2 spike protein (Wuhan strain) with C-terminal His-tag (see FIG. 1).
^{d}Modified S protein trimer from SARS-CoV-2 (aa 1-1208), furin cleavage site removed, C-terminal His-tagged.
^{e}FANA-ST: starting material for the SELEX procedure (see above under “a”).
^{f}K_{D,app} values were determined using nitrocellulose filter binding in PBS buffer unless otherwise state. Results are averages of 2-3 experiment ± standard deviation (for experiments with 3 determinations only).
^{g}RBD from the Delta variant differs from Wuhan strain RBD at two amino acids: Lys452Arg, Thr478Lys.
^{h}Assays were performed in cell culture media: D-MEM + 10% fetal bovine serum (FBS).
ⁱBinding of S1 protein from SARS-CoV-1 (2003 virus) to FANA-R8-9.
^{j}DNA aptamer from (36).

SEQUENCES
Full Sequences from FIGure 2A (First 20-nts are DNA,
the rest are FANA nts, ~40 nucleotide
random region underlined):
R8-5
(SEQ ID NO: 1)
5′-AAAAGGTAGTGCTGAATTCGUCGAACUAAACCGAAUUGAUGGC
AUAAAAAGCAUGUUUAGUUCGCUAUCCAGUUGGCCU-3′
R8-5 Random Region
(SEQ ID NO: 2)
5′-UCGAACUAAACCGAAUUGAUGGCAUAAAAAGCAUGUUUAG-3′
R8-17
(SEQ ID NO: 3)
5′-AAAAGGTAGTGCTGAATTCGUCGAACUAAUCCGAAUUGAUGGCAUAAAAAGC
AUGUUUAGUUCGCUAUCCAGUUGGCCU-3′
R8-17 Random Region
(SEQ ID NO: 4)
5′-UCGAACUAAUCCGAAUUGAUGGCAUAAAAAGCAUGUUUAG-3′
R8-28
(SEQ ID NO: 5)
5′-AAAAGG
TAGTGCTGAATTCGACGAACUAAUCCGAAUUGAUGGCAUAAAAAGCAUG
UUUAGUUCGCUAUCCAGUUGGCCU-3′
R8-28 Random Region
(SEQ ID NO: 6)
5′-ACGAACUAAUCCGAAUUGAUGGCAUAAAAAGCAUGUUUAG-3′
R8-16
(SEQ ID NO: 7)
5′-AAAAGGT
AGTGCTGAATTCGUCGAAACAAUCCGAAUUGAUGGCAUAAAAAGCAUG
AUUAGUUCGCUAUCCAGUUGGCCU-3′
R8-16 Random Region
(SEQ ID NO: 8)
5′-UCGAAACAAUCCGAAUUGAUGGCAUAAAAAGCAUGAUUAG-3′
R8-23
(SEQ ID NO: 9)
5′-AAAAGGTAG
TGCTGAATTCGACUAACUAUUGAUAAUUGAUGGCAUAUAAAACCUG
AUGAAUUCGCUAUCCAGUUGGCCU-3′
R8-23 Random Region
(SEQ ID NO: 10)
5′-ACUAACUAUUGAUAAUUGAUGGCAUAUAAAACCUGAUGAA-3′
R8-9
(SEQ ID NO: 11)
5′-AAAAGGTAG
TGCTGAATTCGCGAGCCCGCAUGAAAAGGGGAGAUAAAAAAUAUCU
GUCGAUUCGCUAUCCAGUUGGCCU-3′
R8-9 Random Region
(SEQ ID NO: 12)
5′-AGCCCGCAUGAAAAGGGGAGAUAAAAAAUAUCUGUCGA-3′
R8-10
(SEQ ID NO: 13)
5′-AAAAGGT
AGTGCTGAATTCGCGAGCCCGCAUGAAAAGGGGAGAUAACAAAUAUCU
GUCGAUUCGCUAUCCAGUUGGCCU-3′
R8-10 Random Region
(SEQ ID NO: 14)
5′-CGAGCCCGCAUGAAAAGGGGAGAUAACAAAUAUCUGUCGA-3′
R8-12
(SEQ ID NO: 15)
5′-AAAAGGT
AGTGCTGAATTCGUGAGCCCGCAUGAAAAGGGGAGAUAAAAAAUAUCU
GUCGAUUCGCUAUCCAGUUGGCCU-3′
R8-12 Random Region
(SEQ ID NO: 16)
5′-UGAGCCCGCAUGAAAAGGGGAGAUAAAAAAUAUCUGUCGA-3′
R8-22
(SEQ ID NO: 17)
5′-AAAAGG
TAGTGCTGAATTCGCGAGCCCGCAUGAAAAGGGGAGAUAUGCAAUAUCU
GUCGAUUCGCUAUCCAGUUGGCCU-3′
R8-22 Random Region
(SEQ ID NO: 18)
5′-CGAGCCCGCAUGAAAAGGGGAGAUAUGCAAUAUCUGUCG-3′
5′-fixed Primer Region
(SEQ ID NO: 19)
5′-AAAAGGTAGTGCTGAATTCG-3′
3′-fixed Primer Region
(SEQ ID NO: 20)
5′-AUUCGCUAUCCAGUUGGCCU-3′
FANA Aptamer Sequence
(SEQ ID NO: 21)
5′-AAAAGGTAGTGCTGAATTCG(N)₄₀AUUCGCUAUCCAGUUGGCCU-3′
Deletion modifications tested for binding to Wuhan
S1 Receptor Binding Domain (RBD):
R8-9-A14-5′
(Binding affinity was essentially the same as R8-9 in Table 1)
(SEQ ID NO: 22)
5′-AATTCGCG
AGCCCGCAUGAAAAGGGGAGAUAAAAAAUAUCUGUCGAUUCGCUAUC
CAGUUGGCCU-3′
R8-9-A10-3′
(Binding affinity was essentially the same as R8-9 in Table 1)
(SEQ ID NO: 23)
5′-AAAAG
GTAGTGCTGAATTCGCGAGCCCGCAUGAAAAGGGGAGAUAAAAAAUAUCU
GUCGAUUCGCUAUC-3′
R8-9-A14-5′-A10-3′
(Binding affinity was essentially the same as R8-9 in
Table 1)
(SEQ ID NO: 24)
5′-AATTCGC
GAGCCCGCAUGAAAAGGGGAGAUAAAAAAUAUCUGUCGAUUCGCUAUC
-3′
R8-17-A14-5′
(Binding affinity was essentially the same as R8-17
in Table 1)
(SEQ ID NO: 25)
5′-AATTCGU
CGAACUAAUCCGAAUUGAUGGCAUAAAAAGCAUGUUUAGUUCGCUAUC
CAGUUGGCCU-3′
R8-17-A14-5′-A10-3′
(Binding affinity was essentially the same as R8-17 in
Table 1)
(SEQ ID NO: 26)
5′-AATTCGU
CGAACUAAUCCGAAUUGAUGGCAUAAAAAGCAUGUUUAGUUCGCUAUC-3′

### REFERENCES

1. Perlman, S. Another Decade, Another Coronavirus. N. Engl. J. Med. 2020, 382, 760-762. [CrossRef]
2. Civljak, R.; Markotic, A.; Kuzman, I. The third coronavirus epidemic in the third millennium: Whats next? Croat. Med. J. 2020, 61, 1-4. [CrossRef]
3. Zhou, P.; Yang, X. L.; Wang, X. G.; Hu, B.; Zhang, L.; Zhang, W.; S1, H. R.; Zhu, Y.; Li, B.; Huang, C. L.; et al. A pneumonia outbreak associated with a new coronavirus of probable bat origin. Nature 2020, 579, 270-273. [CrossRef] [PubMed]
4. Hamming, I.; Timens, W.; Bulthuis, M. L.; Lely, A. T.; Navis, G.; van Goor, H. Tissue distribution of ACE2 protein, the functional receptor for SARS coronavirus. A first step in understanding SARS pathogenesis. J. Pathol. 2004, 203, 631-637. [CrossRef]
5. Sims, A. C.; Baric, R. S.; Yount, B.; Burkett, S. E.; Collins, P. L.; Pickles, R. J. Severe acute respiratory syndrome coronavirus infection of human ciliated airway epithelia: Role of ciliated cells in viral spread in the conducting airways of the lungs. J. Virol. 2005, 79, 15511-15524. [CrossRef]
6. Taylor, P. C.; Adams, A. C.; Hufford, M. M.; de la Torre, I.; Winthrop, K.; Gottlieb, R. L. Neutralizing monoclonal antibodies for treatment of COVID-19. Nat. Rev. Immunol. 2021, 21, 382-393. [CrossRef]
7. Jaworski, J. P. Neutralizing monoclonal antibodies for COVID-19 treatment and prevention. Biomed. J. 2021, 44, 7-17. [CrossRef]
8. Marovich, M.; Mascola, J. R.; Cohen, M. S. Monoclonal Antibodies for Prevention and Treatment of COVID-19. JAMA 2020, 324, 131-132. [CrossRef] [PubMed]
9. Prasad, R. Aptamer-based assay developed for coronavirus detection. The Hindu, 13 Jul. 2020.
10. Brody, E. N.; Gold, L. Aptamers as therapeutic and diagnostic agents. J. Biotechnol. 2000, 74, 5-13. [CrossRef]
11. Deisingh, A. K. Aptamer-based biosensors: Biomedical applications. Handb. Exp. Pharmacol. 2006, 173, 341-357.
12. Gold, L. The SELEX process: A surprising source of therapeutic and diagnostic compounds. Harvey Lect. 1995, 91, 47-57. [PubMed]
13. James, W. Aptamers in the virologists' toolkit. J. Gen. Virol. 2007, 88, 351-364. [CrossRef] [PubMed]
14. Joshi, P. J.; Fisher, T. S.; Prasad, V. R. Anti-HIV inhibitors based on nucleic acids: Emergence of aptamers as potent antivirals. Curr. Drug Targets Infect. Disord. 2003, 3, 383-400. [CrossRef]
15. Nimjee, S. M.; Rusconi, C. P.; Sullenger, B. A. Aptamers: An emerging class of therapeutics. Annu. Rev. Med. 2005, 56, 555-583. [CrossRef]
16. Porschewski, P.; Grattinger, M. A.; Klenzke, K.; Erpenbach, A.; Blind, M. R.; Schafer, F. Using aptamers as capture reagents in bead-based assay systems for diagnostics and hit identification. J. Biomol. Screen. 2006, 11, 773-781. [CrossRef]
17. Zhang, Z.; Blank, M.; Schluesener, H. J. Nucleic acid aptamers in human viral disease. Arch. Immunol. Ther. Exp. (Warsz.) 2004, 52, 307-315.
18. Mok, W.; Li, Y. Recent Progress in Nucleic Acid Aptamer-Based Biosensors and Bioassays. Sensors 2008, 8, 7050-7084. [CrossRef]
19. Andreola, M. L.; Pileur, F.; Calmels, C.; Ventura, M.; Tarrago-Litvak, L.; Toulme, J. J.; Litvak, S. DNA aptamers selected against the HIV-1 RNase H display in vitro antiviral activity. Biochemistry 2001, 40, 10087-10094. [CrossRef]
20. de Soultrait, V. R.; Lozach, P. Y.; Altmeyer, R.; Tarrago-Litvak, L.; Litvak, S.; Andreola, M. L. DNA aptamers derived from HIV-1 RNase H inhibitors are strong anti-integrase agents. J. Mol. Biol. 2002, 324, 195-203. [CrossRef]
21. Ferguson, M. R.; Rojo, D. R.; Somasunderam, A.; Thiviyanathan, V.; Ridley, B. D.; Yang, X.; Gorenstein, D. G. Delivery of double-stranded DNA thioaptamers into HIV-1 infected cells for antiviral activity. Biochem. Biophys. Res. Commun. 2006, 344, 792-797. [CrossRef][PubMed]
22. Fisher, T. S.; Joshi, P.; Prasad, V. R. HIV-1 reverse transcriptase mutations that confer decreased in vitro susceptibility to anti-RT DNA aptamer RT1t49 confer cross resistance to other anti-RT aptamers but not to standard RT inhibitors. AIDS Res. Ther. 2005, 2,
8. [CrossRef] [PubMed]
23. Hotoda, H.; Koizumi, M.; Koga, R.; Kaneko, M.; Momota, K.; Ohmine, T.; Furukawa, H.; Agatsuma, T.; Nishigaki, T.; Sone, J.; et al. Biologically active oligodeoxyribonucleotides. 5. 5′-End-substituted d(TGGGAG) possesses anti-human immunodeficiency virus type 1 activity by forming a G-quadruplex structure. J. Med. Chem. 1998, 41, 3655-3663. [CrossRef][PubMed]
24. Joshi, P. J.; North, T. W.; Prasad, V. R. Aptamers directed to HIV-1 reverse transcriptase display greater efficacy over small hairpin RNAs targeted to viral RNA in blocking HIV-1 replication. Mol. Ther. 2005, 11, 677-686. [CrossRef]
25. Kolb, G.; Reigadas, S.; Castanotto, D.; Faure, A.; Ventura, M.; Rossi, J. J.; Toulme, J.-J. Endogenous expression of an anti-TAR aptamer reduces HIV-1 replication. RNA Biol. 2006, 3, 150-156. [CrossRef] [PubMed]
26. Matzen, K.; Elzaouk, L.; Matskevich, A. A.; Nitzsche, A.; Heinrich, J.; Moelling, K. RNase H-mediated retrovirus destruction in vivo triggered by oligodeoxynucleotides. Nat. Biotechnol. 2007, 25, 669-674. [CrossRef]
27. Moelling, K.; Matskevich, A.; Jung, J. S. Relationship between retroviral replication and RNA interference machineries. Cold Spring Harb. Symp. Quant. Biol. 2006, 71, 365-368. [CrossRef]
28. Neff, C. P.; Zhou, J.; Remling, L.; Kuruvilla, J.; Zhang, J.; Li, H.; Smith, D. D.; Swiderski, P.; Rossi, J. J.; Akkina, R. An aptamer-siRNA chimera suppresses HIV-1 viral loads and protects from helper CD4(+) T cell decline in humanized mice. Sci. Transl. Med. 2011, 3, 66ra66. [CrossRef] [PubMed]
29. Zhou, J.; Neff, C. P.; Liu, X.; Zhang, J.; Li, H.; Smith, D. D.; Swiderski, P.; Aboellail, T.; Huang, Y.; Du, Q.; et al. Systemic administration of combinatorial dsiRNAs via nanoparticles efficiently suppresses HIV-1 infection in humanized mice. Mol. Ther. 2011, 19, 2228-2238. [CrossRef] [PubMed]
30. DeStefano, J. J.; Nair, G. R. Novel aptamer inhibitors of human immunodeficiency virus reverse transcriptase. Oligonucleotides 2008, 18, 133-144. [CrossRef]
31. Lai, Y. T.; DeStefano, J. J. DNA aptamers to human immunodeficiency virus reverse transcriptase selected by a primer-free SELEX method: Characterization and comparison with other aptamers. Nucleic Acid Ther. 2012, 22, 162-176. [CrossRef] [PubMed]
32. Michalowski, D.; Chitima-Matsiga, R.; Held, D. M.; Burke, D. H. Novel bimodular DNA aptamers with guanosine quadruplexes inhibit phylogenetically diverse HIV-1 reverse transcriptases. Nucleic Acids Res. 2008, 36, 7124-7135. [CrossRef] [PubMed]
33. Ellington, A. D.; Szostak, J. W. In vitro selection of RNA molecules that bind specific ligands. Nature 1990, 346, 818-822. [CrossRef][PubMed]
34. Tuerk, C.; Gold, L. Systematic evolution of ligands by exponential enrichment: RNA ligands to bacteriophage T4 DNA polymerase. Science 1990, 249, 505-510. [CrossRef][PubMed]
35. Pinheiro, V. B.; Holliger, P. The XNA world: Progress towards replication and evolution of synthetic genetic polymers. Curr. Opin. Chem. Biol. 2012, 16, 245-252. [CrossRef]
36. Pinheiro, V. B.; Taylor, A. I.; Cozens, C.; Abramov, M.; Renders, M.; Zhang, S.; Chaput, J. C.; Wengel, J.; Peak-Chew, S. Y.; McLaughlin, S. H.; et al. Synthetic genetic polymers capable of heredity and evolution. Science 2012, 336, 341-344. [CrossRef]
37. Schmidt, M.; Pei, L. Synthetic toxicology: Where engineering meets biology and toxicology. Toxicol. Sci. 2011, 120, S204-S224. [CrossRef]
38. Zhang, Y.; Wang, Z.; Gemeinhart, R. A. Progress in microRNA delivery. J. Control. Release 2013, 172, 962-974. [CrossRef]
39. Watts, J. K.; Katolik, A.; Viladoms, J.; Damha, M. J. Studies on the hydrolytic stability of 2′-fluoroarabinonucleic acid (2′F-ANA). Org. Biomol. Chem. 2009, 7, 1904-1910. [CrossRef][PubMed]
40. Watts, J. K.; Damha, M. J. 2′F-arabinonucleic acids (2′F-ANA)—History, properties, and new frontiers. Can. J. Chem. 2008, 86, 641-656. [CrossRef]
41. Alves Ferreira-Bravo, I.; Cozens, C.; Holliger, P.; DeStefano, J. J. Selection of 2′-deoxy-2′-fluoroarabinonucleotide (FANA) aptamers that bind HIV-1 reverse transcriptase with picomolar affinity. Nucleic Acids Res. 2015, 43, 9587-9599. [CrossRef]
42. Rose, K. M.; Alves Ferreira-Bravo, I.; Li, M.; Craigie, R.; Ditzler, M. A.; Holliger, P.; DeStefano, J. J. Selection of 2′-Deoxy-2′-Fluoroarabino Nucleic Acid (FANA) Aptamers That Bind HIV-1 Integrase with Picomolar Affinity. ACS Chem. Biol. 2019, 14, 2166-2175. [CrossRef]
43. Huang, Y.; Yang, C.; Xu, X. F.; Xu, W.; Liu, S. W. Structural and functional properties of SARS-CoV-2 spike protein: Potential antivirus drug development for COVID-19. Acta Pharmacol. Sin. 2020, 41, 1141-1149. [CrossRef]
44. Xia, X. Domains and Functions of Spike Protein in SARS-CoV-2 in the Context of Vaccine Design. Viruses 2021, 13, 109.
45. Rozewicki, J.; Li, S.; Amada, K. M.; Standley, D. M.; Katoh, K. MAFFT-DASH: Integrated protein sequence and structural alignment. Nucleic Acids Res. 2019, 47, W5-W10. [CrossRef] [PubMed]
46. RNAfold. Available online: http://ma.tbi.univie.ac.at/cgi-bin/RNAWebSuite/RNAfold.cgi (accessed on 1 Jul. 2021).
47. Li, J.; Zhang, Z.; Gu, J.; Stacey, H. D.; Ang, J. C.; Capretta, A.; Filipe, C. D. M.; Mossman, K. L.; Balion, C.; Salena, B. J.; et al. Diverse high-affinity DNA aptamers for wild-type and B.1.1.7 SARS-CoV-2 spike proteins from a pre-structured DNA library. Nucleic Acids Res. 2021, 49, 7267-7279. [CrossRef]
48. Liu, Z.; Xiao, X.; Wei, X.; Li, J.; Yang, J.; Tan, H.; Zhu, J.; Zhang, Q.; Wu, J.; Liu, L. Composition and divergence of coronavirus spike proteins and host ACE2 receptors predict potential intermediate hosts of SARS-CoV-2. J. Med. Virol. 2020, 92, 595-601. [CrossRef]
49. Song, Y.; Song, J.; Wei, X.; Huang, M.; Sun, M.; Zhu, L.; Lin, B.; Shen, H.; Zhu, Z.; Yang, C. Discovery of Aptamers Targeting the Receptor-Binding Domain of the SARS-CoV-2 Spike Glycoprotein. Anal. Chem. 2020, 92, 9895-9900. [CrossRef] [PubMed]
50. Schmitz, A.; Weber, A.; Bayin, M.; Breuers, S.; Fieberg, V.; Famulok, M.; Mayer, G. A SARS-CoV-2 Spike Binding DNA Aptamer that Inhibits Pseudovirus Infection by an RBD-Independent Mechanism. Angew. Chem. Weinheim Bergstr. Ger. 2021, 133, 10367-10373. [CrossRef]
51. Yang, G.; Li, Z.; Mohammed, I.; Zhao, L.; Wei, W.; Xiao, H.; Guo, W.; Zhao, Y.; Qu, F.; Huang, Y. Identification of SARS-CoV-2-against aptamer with high neutralization activity by blocking the RBD domain of spike protein 1. Signal. Transduct. Target. Ther. 2021, 6, 227. [CrossRef] [PubMed]
52. Sun, M.; Liu, S.; Wei, X.; Wan, S.; Huang, M.; Song, T.; Lu, Y.; Weng, X.; Lin, Z.; Chen, H.; et al. Aptamer Blocking Strategy Inhibits SARS-CoV-2 Virus Infection. Angew.

Chem. Int. Ed. Engl. 2021, 60, 10266-10272. [CrossRef]
53. Wrapp, D.; Wang, N.; Corbett, K. S.; Goldsmith, J. A.; Hsieh, C. L.; Abiona, O.; Graham, B. S.; McLellan, J. S. Cryo-EM structure of the 2019-nCoV spike in the prefusion conformation.

Science 2020, 367, 1260-1263. [CrossRef]
54. Eremeeva, E.; Herdewijn, P. Non canonical genetic material. Curr. Opin. Biotechnol. 2019, 57, 25-33. [CrossRef] [PubMed]
55. Anosova, I.; Kowal, E. A.; Dunn, M. R.; Chaput, J. C.; Van Horn, W. D.; Egli, M. The structural diversity of artificial genetic polymers. Nucleic Acids Res. 2016, 44, 1007-1021. [CrossRef] [PubMed]
56. Kasahara, Y.; Irisawa, Y.; Fujita, H.; Yahara, A.; Ozaki, H.; Obika, S.; Kuwahara, M. Capillary electrophoresis-systematic evolution of ligands by exponential enrichment selection of base- and sugar-modified DNA aptamers: Target binding dominated by 2′-0,4′-C-methylene-bridged/locked nucleic acid primer. Anal. Chem. 2013, 85, 4961-4967. [CrossRef] [PubMed]
57. Trujillo, C. A.; Nery, A. A.; Alves, J. M.; Martins, A. H.; Ulrich, H. Development of the anti-VEGF aptamer to a therapeutic agent for clinical ophthalmology. Clin. Ophthalmol. 2007, 1, 393-402.
58. Lee, J. H.; Canny, M. D.; De Erkenez, A.; Krilleke, D.; Ng, Y. S.; Shima, D. T.; Pardi, A.; Jucker, F. A therapeutic aptamer inhibits angiogenesis by specifically targeting the heparin binding domain of VEGF165. Proc. Natl. Acad. Sci. USA 2005, 102, 18902-18907. [CrossRef][PubMed]
59. Rohloff, J. C.; Gelinas, A. D.; Jarvis, T. C.; Ochsner, U. A.; Schneider, D. J.; Gold, L.; Janjic, N. Nucleic Acid Ligands With Protein-like Side Chains: Modified Aptamers and Their Use as Diagnostic and Therapeutic Agents. Mol. Ther. Nucleic Acids 2014, 3, e201. [CrossRef]
60. Bernetti, M.; Cavalli, A.; Mollica, L. Protein-ligand (un)binding kinetics as a new paradigm for drug discovery at the crossroad between experiments and modelling. MedChemComm 2017, 8, 534-550. [CrossRef]

What is claimed:

1. A 2′-fluoro-arabinonucleic acid (FANA) aptamer that binds to a SARS-CoV-2 receptor binding domain or S1 domain of SARS-CoV-2 and thereby blocks binding of SARS-CoV-2 to the angiotensin-converting enzyme 2 (ACE2) expressed on the surface of a host cell.

2. The FANA aptamer of claim 1, wherein said aptamer comprises a nucleotide sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to the SEQ ID NOs selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 and SEQ ID NO: 18, or a fragment thereof.

3. The FANA aptamer of claim 2, wherein said aptamer comprises a nucleotide sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 and SEQ ID NO: 18, or a fragment thereof.

4. The FANA aptamer of claim 2, further comprising:
(i) a fixed primer region 5′-AAAAGGTAGTGCTGAATTCG-3′ (SEQ ID NO: 19) at the 5′ end of the aptamer and a fixed primer region 5′-UUCGCUAUCCAGUUGGCCU-3′ (SEQ ID NO: 20) at the 3′ end at the aptamer, or fragments thereof, or
(ii) a fixed primer region at the 5′ end of the aptamer having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% to SEQ ID NO; 19 and a fixed primer region at the 3′ end of the aptamer having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% (SEQ ID NO: 20), or, fragments thereof.

5. The FANA aptamer of claim 3, further comprising:
(i) a fixed primer region 5′-AAAAGGTAGTGCTGAATTCG-3′ (SEQ ID NO: 19) at the 5′ end of the aptamer and a fixed primer region 5′-UUCGCUAUCCAGUUGGCCU-3′ (SEQ ID NO: 20) at the 3′ end at the aptamer, or fragments thereof, or
(ii) a fixed primer region at the 5′ end of the aptamer having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% to SEQ ID NO; 19 and a fixed primer region at the 3′ end of the aptamer having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% (SEQ ID NO: 20), or fragments thereof.

6. The FANA aptamer of claim 1, wherein said aptamer comprises a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NOs selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 12, SEQ ID NO: 23, SEQ ID NO: 24, SEQ ID NO: 25, and SEQ ID NO: 26, or a fragment thereof.

7. The FANA aptamer of claim 1, wherein said aptamer comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 12, SEQ ID NO: 23, SEQ ID NO: 24, SEQ ID NO: 25, and SEQ ID NO: 26, or a fragment thereof.

8. The FANA aptamer of claim 1, further comprising a modification that increases the stability of the FANA aptamer.

9. The FANA aptamer of claim 1, wherein the FANA nucleotide aptamer is resistant to nuclease degradation.

10. An anti-viral pharmaceutical composition comprising the FANA aptamer of claim 1 and a pharmaceutically acceptable carrier.

11. An anti-viral pharmaceutical composition comprising the FANA aptamer of claim 2 and a pharmaceutically acceptable carrier.

12. An anti-viral pharmaceutical composition comprising the FANA aptamer of claim 3 and a pharmaceutically acceptable carrier.

13. An anti-viral pharmaceutical composition comprising the FANA aptamer of claim 4 and a pharmaceutically acceptable carrier.

14. The anti-viral pharmaceutical composition of claim 10, formulated for intranasal administration.

15. The anti-viral pharmaceutical composition of claim 10, wherein the FANA nucleotide aptamer is associated with a nanoparticle.

16. The FANA nucleotide aptamer of claim 1, for use in the treatment or prevention of SARS-CoV-2 infection or a variant thereof.

17. A method of treating, or preventing COVID-19, caused by a SARS-CoV-2 viral infection in a subject in need thereof, said method comprising administering to the subject a therapeutically effective amount of a pharmaceutic composition of claim 10.

18. A kit comprising the pharmaceutical composition of claim 10.

19. A method for identifying a FANA aptamer, useful as an anti-viral composition, comprising the steps of (i) incubating a FANA random pool of aptamers to the target of interest; (ii) washing any unbound FANA aptamers from the incubation mixture; and (iii) releasing the bound FANA from the target; (iv) reverse transcription of the released FANA to DNA; (v) conversion of reverse transcribed DNA to a FANA aptamer; and (vi) repeating the selection steps of (i) to (v), wherein the incubation of step (i) is done with the previously selected FANA(s), until a target binding FANA aptamer is identified with high binding affinity to the target.

20. The method of claim 19, wherein the target of interest is a SARS-CoV2 receptor binding domain (RBD) or a viral Spike S1 protein domain.
